SEQUENCE LISTING (14 sequences)

SEQ ID NO: 1 - protein, 6 aa, Artificial sequence
  Synthetic
GAGGVG

SEQ ID NO: 2 - protein, 6 aa, Artificial sequence
  Synthetic
GVGGTP

SEQ ID NO: 3 - protein, 9 aa, Artificial sequence
  Synthetic
GAGGVGGTP

SEQ ID NO: 4 - protein, 4 aa, Artificial sequence
  Synthetic
GRPG

SEQ ID NO: 5 - protein, 618 aa, Artificial sequence
  Synthetic
MGQPFPEQPEQIIPQQPGAGGVGGTPFPQPEQPFPWQQGAGGVGGTPELPYPQPELPYPQPGAGGVGGTPFPQPELPYPQPEGAGGVGGTPQPFPQPELPYPQPGAGGVGGTPEQPIPEQPQPYPQPGAGGVGGTPEQPFPQPEQPFPQQGAGGVGGTPGRPGMDPRLREEVVRLIIALTSDNGASLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQIVNNNLTLKTTVFDSINSRIGATEQSYVASAVTPLRLNSSTKVLDMLIDSSTLEINSSGQLTVRSTSPNLRYPIADVSGGIGMSPNYRFRQSMWIGIVSYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFT

SEQ ID NO: 6 - protein, 637 aa, Artificial sequence
  Synthetic
MGQFRVIGPRHPIRALVGDEVELGAGGVGGTPGMEVGWYRPPFSRVVHLYRNGKDGAGGVGGTPENPVVHFFKNIVTPRTPGAGGVGGTPGVDAQGTLSKIFKLGGRDSRSGSPMAGAGGVGGTPGTEKLIETYFSKNYQDYEGAGGVGGTPHCLGKWLGHPDKFVGITGAGGVGGTPGRPGMDPRLREEVVRLIIALTSDNGASLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQIVNNNLTLKTTVFDSINSRIGATEQSYVASAVTPLRLNSSTKVLDMLIDSSTLEINSSGQLTVRSTSPNLRYPIADVSGGIGMSPNYRFRQSMWIGIVSYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFT

SEQ ID NO: 7 - protein, 569 aa, Artificial sequence
  Synthetic
MFVNQHLCGSHLVEALYLVCGERGFFYTPKTGAGGVGGTPEAEDLQVGQVELGGGPGAGSLQPLALEGSLQGAGGVGGTPGIVEQCCTSICSLYQLENYCNGAGGVGGTPGRPGMDPRLREEVVRLIIALTSDNGASLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQIVNNNLTLKTTVFDSINSRIGATEQSYVASAVTPLRLNSSTKVLDMLIDSSTLEINSSGQLTVRSTSPNLRYPIADVSGGIGMSPNYRFRQSMWIGIVSYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFT

SEQ ID NO: 8 - protein, 13 aa, Arachis hypogaea
SQLERANLRPCEQ

SEQ ID NO: 9 - protein, 11 aa, Arachis hypogaea
ANLRPCEQHLM

SEQ ID NO: 10 - protein, 12 aa, Arachis hypogaea
ELNEFENNQRCM

SEQ ID NO: 11 - protein, 8 aa, Arachis hypogaea
FENNQRCM

SEQ ID NO: 12 - protein, 14 aa, Arachis hypogaea
RELRNLPQQCGLRA

SEQ ID NO: 13 - protein, 47 aa, Artificial sequence
  Synthetic
MRGSHHHHHHGMASMTGGQQMGRDLYDDDDKDHPFTEFGAGGVGGTP

SEQ ID NO: 14 - protein, 49 aa, Artificial sequence
  Synthetic
MRGSHHHHHHGMASMTGGQQMGRDLYDDDDKDHPFTEFLEGAGGVGGTP

(19) United States
(12) Patent Application Publication
(10) Pub. No.: US 2019/0070287 A1
(43) Pub. Date: Mar. 7, 2019

(54) TOLERANCE THERAPEUTIC FOR TREATING POLYPEPTIDE INDUCED IMMUNE ACTIVATION

(71) Applicant: Virtici, LLC, Seattle, WA (US)

(72) Inventors: Neil A. Fanger, Mercer Island, WA (US); Paul Hill, Snohomish, WA (US); Stacy Martin, Woodinville, WA (US)

(73) Assignee: Virtici, LLC, Seattle, WA (US)

(21) Appl. No.: 16/077,267

(22) PCT Filed: Feb. 10, 2017
(86) PCT No.: PCT/US2017/017346

§ 371 Date: Aug. 10, 2018

Related U.S. Application Data
(60) Provisional application No. 62/294,934, filed on Feb. 12, 2016.

(51) Int. Cl. A61K 39/35; A61K 39/00; A61P 37/06; C12N 15/62; C07K 14/005

(52) U.S. Cl. CPC A61K 39/35; A61K 39/0008; A61K 2039/70; C12N 15/62; C07K 14/005; A61P 37/06

(57) ABSTRACT

The present disclosure is directed to compositions comprising one or more components of a polypeptide, a reovirus-derived targeting protein, and a cleavable linker, and related methods and compositions for the generation of tolerance against the polypeptide. In some embodiments, the polypeptide is antigenic, such as comprising at least one epitope from a food allergen, an environmental allergen, an auto-antigen, and/or a biological therapeutic, and/or at least one epitope derived therefrom.

### CROSS-REFERENCE TO RELATED APPLICATION

This application claims the benefit of U.S. Provisional Patent Application No. 62/294,934 filed Feb. 12, 2016, the disclosure of which is incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 57888_Sequence_final_2017-02-09.txt. The text file is 19 KB; was created on Feb. 9, 2017; and is being submitted via EFS-Web with the filing of the specification.

### FIELD OF THE INVENTION

The present disclosure is directed to the generation of tolerance against polypeptide antigens using a fusion protein comprising at least one antigenic peptide, a reovirus-derived targeting protein, and at least one cleavable linker.

### BACKGROUND

Induction of tolerance to polypeptide antigens can be desirable to avoid inappropriate immune responses. Many studies address the development of tolerance to target antigens by causing the display of reactive epitopes representing the indicated polypeptide antigens in the MHC of a subset of microfold cells (M cells), which are specialized antigen presenting cells of the epithelial layer in the gut and nasopharyngeal associated lymphoid tissue. However, induction of tolerance can be confounded by the inability to specifically target the representative epitopes to the appropriate M cell population, as well as limitations of the M cell's internal processing of polypeptide antigen for incorporation into the MHC presentation.

To illustrate the need to induce tolerance to target polypeptides, it is noted that allergies against proteins found in foods and environmental factors are a major health concern worldwide. An allergy is a hypersensitivity of the immune system to particular antigens (also referred to as “allergens”), which can result in uncomfortable and potentially dangerous immune reactions that can cause severe swelling, rhinitis, bronchoconstriction, edema, hypotension, digestive distress, hives, and itchy sensations. The range of severity can vary greatly from mere discomfort, to inducement of vomiting, asphyxiation, coma and even death.

Potential allergens can be derived from a variety of sources, such as food, plants, chemicals and environmental antigens. Strategies to address allergies include avoidance of the allergen, induction of tolerance (i.e., preventing the hypersensitive reaction when exposed), and ameliorating the response once it occurs.

As one example of the breadth and severity of allergies in a population, it is estimated that more than 1% of the US population (˜3 million people) suffer from peanut or tree nut allergies. Approximately half of the 30,000 food allergy-related emergency room visits each year, including 100-150 deaths, are due to peanut allergies. Unlike many food allergies, reaction to peanuts persists throughout adulthood in approximately 80% of individuals. Taken together, these numbers indicate that peanut allergy represents the most prevalent and severe form of food allergy. Although there has been progress in developing oral desensitization procedures for peanut allergies, the regimens require a gradual increase in exposure over approximately 12 months or more and are not applicable to severely allergic individuals because of potential anaphylactic responses. In addition, the responses observed with these existing oral desensitization regimens are not long-lasting, and the patients' allergic response to peanut allergy returns shortly after stopping the oral administration of allergen. As a result, the vast majority of sufferers rely on strict avoidance and epinephrine administration if exposed. However, because peanuts are such a common food source, the risk of exposure is always a concern, particularly in children.

In addition to allergen polypeptides, immune responses to autoantigens and protein therapeutics also result in a variety of adverse medical conditions and prevent efficient use of therapeutic strategies in healthcare.

Accordingly, a need remains for a simple and effective approach to address inappropriate immune responses to various polypeptides, such as autoantigens, polypeptide allergens, and biological therapeutics. The present disclosure addresses this and related needs by providing a strategy to induce tolerance to polypeptide allergens by incorporating in a fusion protein at least one antigenic peptide of the allergen, a reovirus-derived targeting protein, and at least one cleavable linker.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic representation of an exemplary fusion protein that includes a tolerogen/antigen and pσ1 targeting polypeptide (shaft and head), and a 6 histidine-tag for purification.

FIGS. 2A and 2B are representative images of pσ1 fusion proteins expressed and purified from E. coli. FIG. 2A is an SDS-PAGE and western blot analysis of VTC-GT1 lanes 2-3, (predicted MW: 70.3 kDa), compared to purified pσ1, lane 1, (predicted MW: 51 kDa). FIG. 2B is an SDS-PAGE analysis of VTC-MS1, lane 1 (predicted MW: 72.7 kDa). Molecular weight marker sizes in kDa are noted in the figures.

FIG. 3 is a representative HPLC-SEC analysis SEC profile of a purified pσ1 fusion protein, VTC-GT1 (0.7 mg/mL, PBS). One peak was found. SEC elution time indicated in minutes, with peak intensity given in arbitrary units (AU).

FIG. 4 is a histogram representation of pσ1 fusion protein efficacy as measured by HeLa and L-cell binding and analyzed by FACS analysis. HeLa and L-cells were incubated with pσ1 (top row, control) VTC-MS1 (center row), or VTC-GT1 (lower row) in staining buffer, followed by incubation with rabbit anti-pσ1 polyclonal antibodies (right peak for all panels) or serum control (left peak for all panels). Cells were detected using anti-rabbit IgG.

### DETAILED DESCRIPTION

The present disclosure is generally directed to tolerance therapeutics and related methods that can induce tolerance to polypeptides that can otherwise induce inappropriate immune responses, such as allergens or select self-antigens.

The gut and the nasopharynx constitute major regions of the body that first contact many antigens and allergens from the environment, such as food-borne or ambient, air-borne allergens. The epithelial layer that covers the Gut Associated Lymphoid Tissue (GALT) and Nasopharyngeal Associated Lymphoid Tissue (NALT) regions contains a subpopulation of microfold cells (M cells) specialized to sample environmental antigens and present them to the adjacent immune cells. A number of studies now indicate that the M cells in the GALT and NALT regions play an important role in the generation of either an immune response or a tolerance response to a given antigen.

Reoviruses are segmented, double-stranded RNA viruses that infect humans via mucosal surfaces and can cause both enteric and respiratory infections. To initiate infection, it has been demonstrated that reoviruses first bind to the surface of M cells. Specifically, a reovirus cell adhesin protein, protein sigma (“pσ1”), has been shown to interact with at least two host receptors via separate binding domains. The head domain binds with a component of tight junctions, whereas sequences contained within the fibrous tail domain bind terminal σ-linked sialic acid residues on host cells.

In view of the above, preliminary studies have been performed to assess the ability of reovirus attachment proteins, such as pσ1, to serve as targeting proteins to assist the delivery of antigenic payloads to M cells. For example, it has been demonstrated that administration of a recombinant fusion protein combining the reovirus pσ1 protein with the full ovalbumin (OVA) protein (OVA-pσ1) reduced OVA induction of serum Ig, IFN-gamma, IL-2 and IL-17 levels, while increasing IL-10 and IL-4 in an IL-10 dependent fashion. Imaging studies demonstrated that the OVA-pσ1 specifically binds to the mucosa surface. Immune cells isolated from the mice were characterized, revealing an induction of anti-inflammatory cytokines and an increase of suppressive regulatory T-cells (Tregs) even with a single dose of OVA-pσ1 fusion protein. See Rynda, A., et al., “Low-dose tolerance is mediated by the microfold cell ligand, reovirus protein sigmal,” J. Immunol. 180:5187-5200 (2008); and Suzuki, H., et al., “Ovalbumin-protein sigma 1 M-cell targeting facilitates oral tolerance with reduction of antigen-specific CD4+ T cells,” Gastroenterology 135:917-925 (2008), each incorporated herein by reference in its entirety. In additional studies, the OVA-pσ1 was further modified to include antigens, i.e., proteolipid protein (PLP) and myelin oligodendrocyte glycoprotein (MOG), that normally induce an autoimmune reaction, EAE, in a murine model. The incorporation of these self-antigens into an OVA-pσ1 construct, either by addition to/or replacement of the OVA component, results in diminished EAE. These results indicate the potential use of the pσ1 to treat autoimmune diseases. See Rynda, A., et al., “IL-28 supplants requirement for T(reg) cells in protein sigmal-mediated protection against murine experimental autoimmune encephalomyelitis (EAE),” PLoS One 5:e8720 (2010); and Rynda-Apple, A., et al., “Active immunization using a single dose immunotherapeutic abates established EAE via IL-10 and regulatory T cells,” Eur. J. Immunol. 41:313-323 (2011), each incorporated herein by reference in its entirety.

However, these preliminary studies are limited to using whole OVA antigen and/or MOG antigen fused to pσ1 to induce tolerance to these specific antigens in specially designed murine models. These studies do not address whether the reovirus pσ1 can be fused generally to any antigenic polypeptide (including other intact whole polypeptide antigens, such as allergens, autoantigens, and biological therapeutics, or protein fragments and derivatives thereof) to effectively induce tolerance to the source of that allergenic polypeptide. Therefore, these studies do not inform as to whether the reovirus pσ1 can be used generally to target food-borne or air-borne protein allergens, autoantigens, biological therapeutic to the M cells and functionally induce tolerance in such a way as to ameliorate a subject's immune reactions to normal exposure of the protein source. The prior studies also do not instruct as to what structural characteristics of the intended polypeptide antigen are required to actually obtain some level of tolerance. For example, is the full-length antigen/allergen required, or can the pσ1-based fusion protein incorporate only a fragment of the full-length antigen/allergen. If so, what fragment(s) is/are preferred for optimized tolerance induction? Can multiple fragments be incorporated in the fusion for enhanced effect? What fragment(s) is/are preferred for optimized protein expression from a cell expression system? What fragment(s) is/are preferred for optimized protein solubility for therapeutic administration? Can the performance of the fusion protein be modulated by inserting and/or manipulating a polypeptide linker? Can the performance of the fusion protein be improved by selectively designing fusion proteins that incorporate polypeptides (or polypeptide fragments) to provide a multivalent fusion protein against distinct polypeptides? If so, what design format is preferable? Is glycosylation of the antigenic polypeptides crucial to the induction of tolerance? Also, which specific combinations of full length or polypeptide fragments are required to effectively treat an individual? Such questions require additional characterization of the reovirus fusion proteins to establish their utility as tolerance-inducing platform.

To address the extensive morbidity associated with hypersensitivity to polypeptide antigens, such as allergens (such as, e.g., peanut or gluten allergens), autoantigens, and biological therapeutics, the present disclosure addresses studies that provide new insight into reagents and therapeutic approaches that efficiently induce tolerance to polypeptide allergens.

In accordance with the foregoing, the present disclosure provides an isolated fusion protein comprising a reovirus-derived targeting polypeptide, at least one antigenic polypeptide, and at least one cleavable linker.

As a preliminary matter, as used herein the terms “protein” and “polypeptide” generally refer to a macromolecule of multiple amino acids linked by peptide (amide) bonds. As used herein, an “amino acid” refers to any of the naturally occurring amino acids found in proteins, D-stereoisomers of the naturally occurring amino acids (e.g., D-threonine), unnatural amino acids, and chemically modified amino acids. Each of these categories of amino acids is not mutually exclusive. α-Amino acids comprise a carbon atom to which is bonded an amino group, a carboxyl group, a hydrogen atom, and a distinctive group referred to as a “side chain.” The side chains of naturally occurring amino acids are well-known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine).

The following abbreviations are typically used for the 20 canonical, naturally occurring canonical amino acids: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Noncanonical amino acids (that is, those that are not naturally found in proteins) are also known in the art, as set forth in, for example, Mol. Cell. Biol., 9:2574 (1989); J. Amer. Chem. Soc., 112:4011-4030 (1990); J. Amer. Chem. Soc., 56:1280-1283 (1991); J. Amer. Chem. Soc., 113:9276-9286 (1991), each reference incorporated herein in its entirety. β- and γ-amino acids are known in the art and are also contemplated herein as noncanonical amino acids. Several methods are known in the art for incorporating noncanonical (or non-naturally-occurring) amino acid residues into proteins. For example, an in vitro system can be employed wherein nonsense mutations are suppressed using chemically aminoacylated suppressor tRNAs. Methods for synthesizing amino acids and aminoacylating tRNA are known in the art.

The polypeptide can also have chemically modified amino acids, which refers to an amino acid whose side chain has been chemically modified. For example, a side chain may be modified to comprise a signaling moiety, such as a fluorophore or a radiolabel. A side chain may be modified to comprise a new functional group, such as a thiol, carboxylic acid, or amino group. Post-translationally modified amino acids are also included in the definition of chemically modified amino acids.

Finally, persons of ordinary skill in the art will readily appreciate that the polypeptide can encompass altered polymer structures, such as a type of peptidomimetic where a canonical chemical aspect of the polypeptide is modified. As used herein, the term “peptidomimetic” refers to compounds whose essential elements (pharmacophore) mimic a natural peptide or polypeptide in 3D space, and which retain the ability to interact with the biological target (e.g., a receptor) and produce the same biological effect as an unmodified, canonical polypeptide structure. However, peptidomimetics are designed to circumvent some of the problems associated with a natural peptide: e.g., stability against proteolysis (duration of activity) and poor bioavailability. Certain other properties, such as receptor selectivity or potency, often can be substantially improved. The structural modifications that result in peptidomimetics are well-known and have been described elsewhere. See, e.g., Vagner, J., et al., “Peptidomimetics, a synthetic tool of drug discovery,” Curr. Opin. Chem. Biol. 12(3):292-296 (2008), incorporated herein by reference in its entirety.

As used herein, the term “isolated” in the context of an isolated fusion protein, indicates that the fusion protein has been produced through human intervention and has been substantially separated from the materials co-existing in the protein production environment, such as the intra-cellular organelles and proteins in a cell culture system. In contrast, a naturally expressed protein in cell is not “isolated.” Furthermore, the term “fusion” in the context of a fusion protein indicates that the overall protein or polypeptide contains a nonnaturally occurring polypeptide sequence. Typically, a fusion protein combines to two or more existing polypeptides or polypeptide fragments, from the same or different source proteins, in a chimeric polymer where the polypeptides (or fragments) do not naturally occur together in that manner. Methods of producing fusion proteins are well known. For example, nucleic acids encoding the different polypeptide components of the fusion protein can be generated and amplified using PCR and assembled into an expression vector in the same reading frame to produce a fusion gene. The expression vector can be transformed into any appropriate expression system, such as prokaryotic or eukaryotic cells, which can then express the protein. See, e.g., such standard references as Coligan, Dunn, Ploegh, Speicher and Wingfield “Current Protocols in Protein Science” (1999), Volume I and II (John Wiley & Sons Inc.); Sambrook et al., “Molecular Cloning: A Laboratory Manual” (1989), 2^{nd} Edition (Cold Spring Harbor Laboratory Press); and Prescott, Harley and Klein “Microbiology” (1999), 4^{th} Edition (WBC McGraw-Hill), each incorporated herein by reference. One exemplary approach for creating fusion proteins is described in more detail in the below examples. In another embodiment, the fusion protein can be created by linking two or more existing polypeptide fragments. For example, the reovirus-derived targeting polypeptide component (e.g., sigma polypeptide (pσ1), homologs thereof, or functional portions thereof as described below) can be produced separately from the antigenic polypeptide(s). Each of these separate components can be generated or obtained independently from one another by any known and conventional technique. The components can subsequently be fused or linked to one another by chemical means. For example, distinct components can have complementary linker components such that they will form strong mutual bonds, thereby linking their respective components to produce the fusion protein. The linker moieties can be homobifunctional or heterobifunctional. An illustrative, nonlimiting example of such chemical linker constructs include having one component (e.g., targeting polypeptide component) include biotin and the other component (e.g., a polypeptide) include strep-avidin, or vice versa. The biotin and strep-avidin moieties will form high-affinity bonds, thereby linking, or “fusing”, the components to result in the fusion protein. Other common linking chemistries can also be used, such as, for example, gluteraldehyde, and the like.

The reovirus-derived targeting polypeptide component of the fusion protein can comprise the reovirus protein sigma polypeptide (pσ1), homologs thereof, or functional portions or derivatives thereof. As used in this context, the term “functional” refers to the ability for the one or more combined portions of the pσ1 polypeptide to induce some degree of tolerance to an antigenic polypeptide fused thereto. Without being bound to any particular theory, this functionality likely requires the ability of the one or more combined portions of the pσ1 polypeptide to bind to the target M cells in the mucosa sufficiently to transfer the antigenic polypeptide thereto. The structure and sequence of the reovirus has been previously described. See, e.g., Turner, D. L., et al., “Site-directed mutagenesis of the C-terminal portion of reovirus protein sigma 1: evidence for a conformation-dependent receptor binding domain,” Virology 186:219-227 (1992); Nibert, M. L., et al., “Infectious subvirion particles of reovirus type 3 Dearing exhibit a loss in infectivity and contain a cleaved sigma 1 protein,” J. Virol. 69:5057-5067 (1995); and Lee, P. W. and Leone, G., “Reovirus protein sigma 1: from cell attachment to protein oligomerization and folding mechanisms,” Bioessays 16:199-206 (1994); Barton, E. S., et al., “Utilization of Sialic Acid as a Coreceptor Enhances Reovirus Attachment by Multistep Adhesion Strengthening,” J. Biol. Chem. 276:2200-2211 (2000); Fraser, R. D. B., et al., “Molecular Structure of the Cell-Attachment Protein of Reovirus: Correlation of Computer-Processed Electron Micrographs with Sequence-Based Predictions,” J. Virol. 64:2990-3000 (1990); Nibert, M. L., et al. “Structure of the Reovirus Cell-Attachment Protein: A Model for the Domain Organization of pS1,” J. Virol. 64:2976-2989 (1990), each of which is incorporated herein by reference in its entirety.

The reovirus-derived targeting polypeptide component can include less than the full length of pσ1 polypeptide, but can contain functional fragments or derivatives of fragments, or fusions of non-contiguous fragments thereof, so long as the protein retains the ability to target the overall fusion protein to M-cells and induce some degree of tolerance to the antigenic polypeptide fused thereto. Domains of the pσ1 that contribute the targeting functionality of the fusion protein include (from C-terminus to N-terminus) the head domain, the trimerization domain, the sialic acid binding domain, and the shaft domain. Although a truncated pσ1 could be constructed, the truncated pσ1 would preferentially still comprise the head domain, which binds with a component of tight junctions on cells, as well as the sequences contained in the tail domain, which bind terminal α-linked sialic acid residues on host cells. These components are typically required for the induction of tolerance. (Zlotkowska, D., et al., “Loss of Sialic Acid Binding Domain Redirects Protein σ1 to Enhance M Cell-Directed Vaccination,” PLoS One 7:e36182 (2012)). Typically, fusions will incorporate the chosen polypeptide(s) at the C-terminal end of the pσ1 polypeptide (or fragment thereof) so as to avoid interfering with the ability of the head domain to bind to the mucosal cell receptors.

As used herein, the term “derivative thereof” refers to any pσ1 protein or functional portion thereof that has one or more amino acid additions, substitutions, or deletions, with respect to a reference pσ1 protein or functional portion thereof that has substantially equivalent or enhanced functionality. For instance, the pσ1 could incorporate various mutations from a reference pσ1 sequence, such as in the head domain that increases the binding avidity of the pσ1 or functional portion thereof to the M cell.

The fusion protein also comprises at least one antigenic polypeptide. In this context, the antigenic polypeptide is any stretch of contiguous amino acids in a polypeptide molecule that stimulates an immune response in a vertebrate, where the immune response has a negative impact on the health, comfort, and well-being of the vertebrate subject. The polypeptide can be the full-length protein of a known allergen, autoantigen, or biological therapeutic. Alternatively, the polypeptide can be “derived from” a source allergen, autoantigen, or biological therapeutic. In this regard, the term “derived from” indicates that the antigenic polypeptide component of the fusion protein can be the result of some processing of the source antigen protein. For example, the antigenic polypeptide can be a fragment of the source protein where one or more end portions of the full-length source proteins have been removed. In another embodiment, the antigenic polypeptide can itself be a fusion of non-contiguous sections of the source antigenic protein, where an internal portion(s) have been removed. It will be appreciated that the remaining portions of the source protein can be oriented in the antigenic polypeptide in a contiguous orientation, or, alternatively, can be separated by a linker moiety (described in more detail below).

In another embodiment, the fusion protein comprises a plurality (i.e., more than one) antigenic polypeptides. In this context, reference to multiple fusion polypeptides as distinct components can imply that the polypeptides are, or are derived from, distinct source proteins or distinct domains of the same source protein. The source proteins themselves may be from: the same protein (e.g., different domains of the same protein), the same overall source (e.g., two distinct source proteins from a peanut), or from different sources (e.g., a source protein from peanut and a source protein from walnut, fish, gluten, dust mites, and the like). The plurality of antigenic polypeptides can be in any relative orientation, including being N-terminal or C-terminal to the pσ1 component of the fusion protein, or chemically linked through an amino acid side chain, as described above.

The multiple components of the fusion protein (e.g., the targeting polypeptide, or subcomponents thereof, e.g., domains of pσ1, and the one or more antigenic polypeptides, and potential subcomponents thereof) can be generally disposed in adjoining, contiguous sequence. However, at least two of the multiple components are joined by a cleavable linker moiety, which would be disposed between the at least two components and covalently attached to each. The presence of a cleavable linker facilitates the processing of the antigenic polypeptide component(s) within the target cell. For example, in instances where a fusion protein includes multiple allergenic polypeptides in a linear sequence, the fusion protein may be resistant to protease digestion. Accordingly, any single allergen polypeptide may be less likely to be properly processed and presented in the context MHC molecules to induce T regulatory cells.

To overcome this obstacle, at least one cleavable linker is introduced between two proximate antigenic polypeptide components and/or between an antigenic polypeptide component and the targeting polypeptide. For example, the cleavable linker can comprise a protease cleavage sequence motif. It will be appreciated that any known cleavage sequence motif is contemplated for this purpose, although a person of ordinary skill in the art will understand that the sequence motif would ideally not interfere with the proper folding of other components of the fusion protein, such as the targeting polypeptide, so as to avoid interfering with the targeting functionality of the fusion protein. In some embodiments, the protease motif is recognized (and cleaved) by a protease that is primarily intracellular in the target cell. For example, an exemplary cleavable linker can comprise any protease cleavage sites for a cathepsin protease, which are well-known in the art. Several cathepsin classes, and their respective target cleavage sites, are known (i.e., Cathepsin A, B, C, D, E, F, G, H, K, L1, L2 (or V), O, S, W and Z). With the exception of cathepsin K, most cathespsin are primarily active intracellularly and, thus, their target cleavage sites are well-suited for incorporation into the cleavable linker described herein. In some embodiments, the cleavable linker comprises cleavage sites for cleavage by, for example, for cathepsin B and/or for cathepsin S. In this regard, both cathepsin B and cathepsin S are expressed in a variety antigen presenting cells. In addition, like most cathepsins, cathepsin B and cathepsin S are not abundantly found in the lumen of the gastrointestinal tract, thus avoiding degradation while in the GI lumen, but facilitate processing of the fusion protein upon cellular internalization by the target cell. An exemplary cathepsin B cleavage site is the amino acid sequence GAGGVG (SEQ ID NO:1). An exemplary cathepsin S cleavage site is the amino acid sequence GVGGTP (SEQ ID NO:2). Thus, exemplary embodiments can comprise either SEQ ID NO:1 or SEQ ID NO:2 as the cleavable linker component of the fusion protein.

Additionally, the cleavable linker can comprise cleavage sites for multiple proteases. For example, the cleavable linker can comprise the amino acid sequence GAGGVGGTP (SEQ ID NO:3), which combines the cathepsin B and cathepsin S cleavage sites set forth in SEQ ID NOS:1 and 2 in an overlapping configuration. This cleavage site is referred to herein as “cathepsin S, B linker”. See Table 1. Such an embodiment can further enhance the intracellular cleavage and processing of the fusion proteins for presentation in the MHC complex and thus, enhance subsequent inducement of tolerance for the source polypeptide.

Notwithstanding the above discussion of cleavable linkers disposed between multiple antigenic polypeptides within the fusion protein, it will also be understood that in other embodiments, including embodiments with only a single antigenic peptide, placement of a cleavable linker between an antigenic polypeptide and the targeting polypeptide can also be useful to increase likelihood of proper MHC processing of the antigenic peptide. Thus, in some embodiments, the fusion protein comprises a cleavable linker disposed between the antigenic polypeptide and the targeting polypeptide. In some embodiments, the fusion protein comprises multiple cleavable linkers, wherein at least one cleavable linker separates the antigenic polypeptide and a first targeting polypeptide, and one or more additional cleavable linkers separate the first targeting polypeptide from additional targeting polypeptides.

In addition to a cleavable linker, the fusion protein can also comprise a flexible linker moiety disposed between any two proximate components of the fusion protein, for example, between an allergen/autoantigen polypeptide and the targeting polypeptide. Such a flexible linker can be a synthetic polypeptide sequence, which is typically between about four and about 40 amino acids in length. The linker preferably provides an attachment between the otherwise proximate components in the fusion providing sufficient space and flexibility such that each component can freely assume its natural three-dimensional configuration without requiring significant adjustment for the configuration assumed by the proximate component. Accordingly, such linkers are typically designed to avoid significant formation of rigid secondary structures that could reduce the flexibility or distance provided between the proximate components. This, the linker is designed to provide a linear or alpha-helical structure. Such linkers are commonly used and are well-understood in the art. As an illustrative, non-limiting example, the linker can comprise the amino acid sequence GlyArgProGly (SEQ ID NO:4). In other embodiments, the linker is a non-polypeptide chemical linker, as known in the art. For example, as described above, the linker moieties can be homobifunctional or heterobifunctional. Examples include strep-avidin/biotin and crosslinkers, such as thiol or amide-linker systems, as used in antibody technologies.

Exemplary allergens and allergen sources that are useful for the allergen polypeptide are now described. A large number of defined allergens are known to the artisan. Online data bases which provide the approved nomenclature for many known allergens and provide links to known nucleic acid and amino acid sequences are available, including for example, the allergenonline data base provided by the University of Nebraska-Lincoln and the official allergen nomenclature website approved by the World Health Organization and the International Union of Immunological Societies Allergen Nomenclature Subcommittee.

The allergen polypeptide of the present disclosure can be generally a food allergen, an environmental allergen, an autoantigen, and/or a biological therapeutic. Moreover, the allergen polypeptide can be derived from any of the sources in the above categories. In this context, the allergen polypeptide integrated into the fusion protein can be a full-length allergen protein found in the allergen source, or can be a subcomponent, or a fusion of multiple subcomponents, of the full-length protein.

Food allergens (and their general food sources) are well-known and many protein components of each allergen have been identified and characterized. For example, illustrative and non-limiting sources of food allergens include various fruits (such as mango and strawberries), garlic, fish, shellfish, meats, milk, peanuts and other legumes or ground nuts, tree nuts (such as almonds, Brazil nuts, cashews, chestnuts, filberts/hazelnuts, macadamia nuts, pecans, pistachios, pine nuts, and walnuts), soy, oats, gluten, and egg.

To further illustrate, in peanut (Arachis hypogeae) allergy there are approximately twelve proteins identified by reactive serum IgE that are, thus, identified as being allergenic. These are referred to by the following abbreviations and in parenthesis a subtype designation and/or a general database identifier (GI|), which database identifiers are incorporated herein by reference: Arah2 (0.0201 GI|26245447; 0.0101 GI|31322014), Arah6 (GI|5923742; GI|17225991, Arah1 (GI|1168390; GI|1168391), Arah3 (0.0101 GI|3703107; 0.0201 GI|5712199), Arah4 (renamed Arah3.0201), Arah5 (GI|5902098; GI|43182555; GI|284810529), Arah7 (GI|5931948; GI|158121995), Arah8 (GI|37499626; GI|145904610; GI|169786740; or GI|110676574), Arah9 (0.0101 GI|161087230; 0.0201 GI|161610580), Arah10 (0.0101 GI|113200509; 0.0102 GI|52001239), Arah11 (0.0101 GI|71040655), and Arah12 (0.0101 GI|160623326). Of these, antibodies to Arah1, 2, and 6 are typically detected in more than 80 to 90% of allergic individuals. Using cell based degranulation assays, it has been reported that removal of Arah2 and Arah6 from whole peanut extract (WPE) reduces the antigenicity by 90%. Because these two proteins are closely related (approximately 60% homology), it has been proposed that a therapy generating a robust tolerance response to either protein would be expected to significantly improve the lives of the majority of peanut allergy sufferers. Thus, it is hypothesized herein that Arah2 represents the best single antigen for developing a pσ1 targeted tolerance therapeutic to treat individuals with peanut allergy. However, as described above, the efficacy of any fusion protein can potentially be improved to treat unresponsive patients by adding another one or two other allergen polypeptides with an Arah2 fusion protein, or by developing additional fusion proteins that contain other major peanut allergens, such as for example, Arah1 and Arah6, and using a combination therapy.

In some embodiments, the food allergen is from gluten. Several protein allergens from gluten are known and have been characterized and are encompassed by the present disclosure. For example, the allergen polypeptide can be a prolamin from wheat (Triticum aestimium), barley (Hordeum vulgare), oats (Avena sativa), rye (Secuale cereal), corn (Zea mays) or sorghum (Sorgham bicolor) and can include, for example α-gliadin, β-gliadin, γ-gliadin, ω-gliadin, hordein, secalin, zein, kafirin, avenin; a glutenin, or can be derived therefrom. The prolamin can include any one of the proteins, protein isoforms, or fragments thereof. These are referred to by the following abbreviations and in parenthesis a subtype designation and/or a general database identifier (GI|), which database identifiers are incorporated herein by reference: Triticum aestivium omega 5 gliadin (tria19) GI|73912496, GI|208605344, GI|208605348, GI|208605346, GI|508732623 Triticum aestivium γ gliadin (tria20) GI|508732621, GI|170702, GI|170708, GI|170736, GI|170738, GI|1063270, GI|62484809, GI|508732621; Triticum aestivium α/β gliadin, for example tria2l and tria25 (GI|28347640, GI|21755, GI|21757, GI|21761, GI|170710, GI|170712, GI|170718, GI|170726, GI|170728 and the like); Hordeum vulgare γ hordein 3, for example, horv20 (GI|288709); Secalae cereal γ seculin, for example, secc20 (GI|75198759, GI|75198753); Avena sativa avenin, for example, GI|166555, GI|166553, GI|166557, GI|166551, GI|389616299; Sorghum bicolor kafirin, for example, GI|21174, GI|125167. Zea mays zein, for example, GI|168701, GI|168699, GI|468517, GI|468515, and the like. All data base identifiers are each incorporated herein by reference.

In some embodiments, the food allergen is from milk. Several allergens from milk are known and have been characterized and are encompassed by the present disclosure. For example, the allergen polypeptide can be alpha S1-casein, for example from Bos taurus, GI|162794, and GI|30794348, alpha S2-casein from Bos taurus, for example, bosd10 GI|27806963, β-lactoglobulin from Bos domesticus, for example, bosd5 GI|520; β-casein from Bos taurus, for example, bosd11 GI|942073448, GI|162797, GI|162805, GI|459292; κ-casein from Bos taurus, for example, bosd12 GI|162811, GI|27881412, or can be derived therefrom.

In some embodiments, the food allergen is from egg. Several allergens from egg are known and have been characterized and are encompassed by the present disclosure. For example, the allergen polypeptide can be ovomucoid from Gallus gallus for example gald1 GI|124757, GI|209979542 or gald2 GI|63052, GI|129293, ovotransferrin from Gallus gallus, for example, gald3 GI|757851, GI|1351295, lysozyme from Gallus gallus, for example gald4 GI|126608, GI|212279, GI|63581, livetin (chicken serum albumin) from Gallus gallus, for example gald5 GI|63748, apovitillin, phosvitin, or can be derived therefrom. Fragments of ovalbumin comprising tolerogenic epitopes are also considered a composition of the present disclosure.

In some embodiments, the food allergen is from fish. Several allergens from fish are known and have been characterized and are encompassed by the present disclosure. For example, the allergen polypeptide can be Che ag, Lop pi, Gelatin/Ore a, parvalbumin from ocean perch Sebastes marinus, for example Sebm1.0101 GI|242253959 or Sebm1.0201 GI|242253961; parvalbumin from talapia Orea1, parvalbumin from Pacific pilchard Sardinops sagax Sarsa1.0101 GI|193247972, parvalbumin from rainbow trout Oncirhynechus mykiss oncm1 GI288559140, glyceraldehyde-3-phosphate dehydrogenase from a number of fish species, or can be derived therefrom.

Environmental allergens (and their general sources) are well-known and many protein components of many environmental allergen sources have been identified and characterized. For example, illustrative and non-limiting sources of environmental allergens include mold proteins, pollen from trees, grasses, and ragweed, dust mites, glycoproteins in animal dander (e.g., from cat and dog), in insect stings (e.g., bee and wasp), other animal (e.g., reptile) venoms, and other animal allergens known in the art.

In some embodiments, the environmental allergen is from a house dust mite. Several allergens from dust mites are known and have been characterized and are encompassed by the present disclosure. For example, the allergen polypeptide can be from Dermatophagoides pteronyssinus, including for example, Derp1 through Derp23, from Dermatophagoides farinae, including for example, Derf1 through Derf33; from Euroglyphus maynei, including for example, (Eurm1 (GI|3941388, incorporated by reference herein), Eurm2 (GI|3941386, incorporated by reference herein), Eurm3 (GI|42004421, incorporated by reference herein), Eurm4 (GI|5059164, incorporated by reference herein), Eurm14 (GI|6492307, incorporated by reference herein); from Dermatophagoides microceras, including for example, Derm1 (GI|127205, incorporated by reference herein), or can be derived therefrom.

In some embodiments, the environmental allergen is from a cat (Fells domesticas. Several allergens from cats are known and have been characterized and are encompassed by the present disclosure. For example, the allergen polypeptide can be a secretoglobin such as Feld1 (chain 1 GI|1364212, GI|1364213, GI|163825, GI|1169655, GI|114326420; chain 2 GI|395407, GI|163823, each incorporated by reference herein), a lipocalin such as Feld4 (GI|45775300, incorporated by reference herein), an albumin such as Feld2 (GI|886485, incorporated by reference herein), a cystatin such as Feld3 (GI|17939981, incorporated by reference herein), IgA such as Feld5w, or can be derived therefrom.

Plants that produce allergy inducing pollen are typically anemophilous (i.e., have their pollen dispersed by wind) and include ragweed, oak, birch, hickory, alder, ash, and pecan trees, and summer grasses. In some embodiments, the environmental allergen is from a tree. Several allergens from trees are known and have been characterized and are encompassed by the present disclosure. For example, the allergen polypeptide can be Betv1 (for example, GI|320545, GI|534900, GI|1321716, GI|1321722, each incorporated by reference herein), Betv2 (for example, GI|57830684, GI|66953, each incorporated by reference herein), Betv3 (GI|488605, incorporated by reference herein), Betv4 (GI|809536, incorporated by reference herein), Betv6 (GI|10764491, incorporated by reference herein), or Betv7 (GI|21886603, incorporated by reference herein) from the European White Birch Betula pendula; Alng1 (GI|261407, incorporated by reference herein), or Alng4 (GI|3319651, incorporated by reference herein) from the alder Alnus glutinosa; Frae1 (GI|33327133, GI|56122438, GI|34978692, each incorporated by reference herein) from the European ash Fraxinus excelsior, or can be derived therefrom. In other embodiments, the environmental allergen is from ragweed (Ambrosia artenisiifolia, Ambrosia psilostachya or Ambrosia trifida. Several allergens from ragweed are known and have been characterized and are encompassed by the present disclosure. For example, the allergen polypeptide can be Amba1 through Amba11 (GI|166435, GI|166437, GI|302127812, GI|166411, GI|166443, GI|302127814, GI|302127816, GI|166445, GI|302127824, GI|166447, GI|302127828, GI|416636, GI|291197394, GI|1916292, GI|62249502, GI|62249512, GI|62249470, GI|62249481, GI|62249491, GI|558482540, each incorporated by reference herein) from Ambrosia artenisiifolia; Ambp5 (GI|515953, GI|515955, each incorporated by reference herein) from Ambrosia psilostachya; Ambt5 (GI|17680, incorporated by reference herein) from Ambrosia tirfida, or can be derived therefrom.

As indicated above, the antigenic polypeptide can comprise an autoantigen, an epitope of an autoantigen, or a derivative thereof. Many autoantigens that can cause autoimmune diseases have been identified and characterized and are encompassed by the present disclosure and can be incorporated in a similar manner as described with reference to allergen polypeptides. For example, the autoantigen can be selected from the non-limiting list of a transglutaminase, myelin-associated glycoprotein (MAG; G|I62205282, incorporated by reference herein), CNS-specific myelin oligodendrocyte glycoprotein (MOG; GI|984147, GI|793839, each incorporated by reference herein), myelin basic protein (MBP, GI|1184244, GI|307161, GI|307162, GI|307160, each incorporated by reference herein), proteolipid protein (PLP, GI|41393531, incorporated by reference herein), Zinc transporter-8 (ZnT8, a chain GI|64762489, b chain GI|289803013, GI|289803009, GI|289803007, GI|289803003, each incorporated by reference herein), glutamic decarboxylase 65 (GAD65, GI|352216, incorporated by reference herein), glutamic decarboxylase 67 (GAD67, GI|1352213, GI|385451, each incorporated by reference herein), preproinsulin (GI758088, GI389620191, GI631226408, each incorporated by reference herein), proinsulin, insulin, tyrosine phosphatase-like autoantigen, insulinoma antigen-2 (IA-2; ICA512, PTPRN; GI|2499754, incorporated by reference herein), IA-2b (Phogrin, PTPRN2; GI|47939489, incorporated by reference herein), islet cell antigen-69 (ICA69; GI|20141584, incorporated by reference herein), chromogranin A (GI|180527, incorporated by reference herein), islet amyloid polypeptide (ppIAPP; GI|4557655, incorporated by reference herein), and heat shock protein 60 (hsp60; GI|77702086, incorporated by reference herein), or can be derived therefrom.

Additionally, antigenic polypeptides can be from biological (i.e., protein-based) therapeutic compositions. For example, portions of humanized antibodies such as the CDRs have been shown to elicit immune responses and, thus, the induction of tolerance to such a therapeutic is desired to maintain the utility of such compositions. Another example is recombinant erythropoietin and other cytokines and therapeutic hormones can elicit immune responses. In addition, other therapeutic proteins can elicit immune responses including for example, growth hormone, interferons, monoclonal antibody therapeutic products, for example Remicade®, Humira®, Simboni®, and the like. Accordingly, the antigenic polypeptide can be any of such biological (i.e., proteinaceous) composition, or can be derived therefrom.

Amino acid sequences of illustrative, non-limiting fusion protein constructs that incorporate different exemplary fusion proteins with cleavable linkers are provided in Table 1.

TABLE 1
illustrative fusion protein constructs incorporating cleavable linkers.

| Fusion | Allergy/ | | | SEQ ID |
|---|---|---|---|---|
| name | disorder | Amino acid sequence | Comment | NO: |
| VTC- | Celiac | [MG]*QPFPEQPEQIIPQQPGAGGVGGTPFPQP | A seven T cell | 5 |
| GT1 | Disease | EQPFPWQQGAGGVGGTPELPYPQPELPYPQ | gluten epitope-pσ1 | |
| | | PGAGGVGGTPFPQPELPYPQPEGAGGVGGT | fusion protein; the | |
| | | PQPFPQPELPYPQPGAGGVGGTPEQPIPEQP | N-terminal, seven | |
| | | QPYPQPGAGGVGGTPEQPFPQPEQPFPQQG | T cell gluten | |
| | | 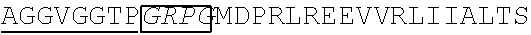 | epitope domain (7 | |
| | | DNGASLSKGLESRVSALEKTSQIHSDTILRIT | gluten epitopes, | |
| | | QGLDDANKRIIALEQSRDDLVASVSDAQLAI | specifically from α- | |
| | | SRLESSIGALQTVVNGLDSSVTQLGARVGQ | gliadin, ω-gliadin, | |
| | | LETGLAELRVDHDNLVARVDTAERNIGSLT | Hordein, and | |
| | | TELSTLTLRVTSIQADFESRISTLERTAVTSA | Secalin, each | |
| | | GAPLSIRNNRMTMGLNDGLTLSGNNLAIRL | separated by a | |
| | | PGNTGLNIQNGGLQFRFNTDQFQIVNNNLT | cleavable cathepsin | |
| | | LKTTVFDSINSRIGATEQSYVASAVTPLRLN | S, B linker | |
| | | SSTKVLDMLIDSSTLEINSSGQLTVRSTSPNL | (underlined)) is | |
| | | RYPIADVSGGIGMSPNYRFRQSMWIGIVSYS | tethered to the | |
| | | GSGLNWRVQVNSDIFIVDDYIHICLPAFDGF | C-terminal pσ1 | |
| | | SIADGGDLSLNFVTGLLPPLLTGDTEPAFHN | domain by a GRPG | |
| | | DVVTYGAQTVAIGLSSGGAPQYMSKNLWV | linker (italics- | |
| | | EQWQDGVLRLRVEGGGSITHSNSKWPAMT | boxed) | |
| | | VSYPRSFT | | |
| VTC- | Multiple | [M]**GQFRVIGPRHPIRALVGDEVELGAGGV | A six T-cell | 6 |
| MS1 | Sclerosis | GGTPGMEVGWYRPPFSRVVHLYRNGKDGA | epitope (from | |
| | | GGVGGTPENPVVHFFKNIVTPRTPGAGGVG | MOG, MBP, and | |
| | | GTPGVDAQGTLSKIFKLGGRDSRSGSPMAG | PLP antigens)-pσ1 | |
| | | AGGVGGTPGTEKLIETYFSKNYQDYEGAGG | fusion protein; the | |
| | | VGGTPHCLGKWLGHPDKFVGITGAGGVGG | N-terminal, six | |
| | | 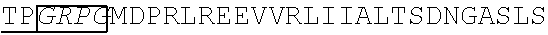 | epitope domain (6 | |
| | | KGLESRVSALEKTSQIHSDTILRITQGLDDA | epitopes, | |
| | | NKRIIALEQSRDDLVASVSDAQLAISRLESSI | specifically from | |
| | | GALQTVVNGLDSSVTQLGARVGQLETGLA | MOG, MBP, and | |
| | | ELRVDHDNLVARVDTAERNIGSLTTELSTLT | PLP antigens each | |
| | | LRVTSIQADFESRISTLERTAVTSAGAPLSIR | separated by a | |
| | | NNRMTMGLNDGLTLSGNNLAIRLPGNTGL | cleavable cathepsin | |
| | | NIQNGGLQFRFNTDQFQIVNNNLTLKTTVF | S, B linker | |
| | | DSINSRIGATEQSYVASAVTPLRLNSSTKVL | (underlined)) is | |
| | | DMLIDSSTLEINSSGQLTVRSTSPNLRYPIAD | tethered to the | |
| | | VSGGIGMSPNYRFRQSMWIGIVSYSGSGLN | C-terminal pσ1 | |
| | | WRVQVNSDIFIVDDYIHICLPAFDGFSIADG | domain by a GRPG | |
| | | GDLSLNFVTGLLPPLLTGDTEPAFHNDVVT | linker (italics- | |
| | | YGAQTVAIGLSSGGAPQYMSKNLWVEQWQ | boxed) | |
| | | DGVLRLRVEGGGSITHSNSKWPAMTVSYPR | | |
| | | SFT | | |
| VTC- | Type 1 | MFVNQHLCGSHLVEALYLVCGERGFFYTPK | A human | 7 |
| DX1 | Diabetes | TGAGGVGGTPEAEDLQVGQVELGGGPGAG | proinsulin-pσ1 | |
| | | SLQPLALEGSLQGAGGVGGTPGIVEQCCTSI | fusion protein; the | |
| | | 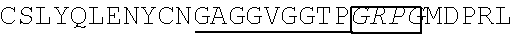 | N-terminal, human | |
| | | REEVVRLIIALTSDNGASLSKGLESRVSALE | proinsulin domain | |
| | | KTSQIHSDTILRITQGLDDANKRIIALEQSRD | (containing human | |
| | | DLVASVSDAQLAISRLESSIGALQTVVNGLD | pro-insulin | |
| | | SSVTQLGARVGQLETGLAELRVDHDNLVA | peptides A, B, and | |
| | | RVDTAERNIGSLTTELSTLTLRVTSIQADFES | D, each separated | |
| | | RISTLERTAVTSAGAPLSIRNNRMTMGLND | by a cleavable | |
| | | GLTLSGNNLAIRLPGNTGLNIQNGGLQFRFN | cathepsin S, B | |
| | | TDQFQIVNNNLTLKTTVFDSINSRIGATEQS | linker (underlined) | |
| | | YVASAVTPLRLNSSTKVLDMLIDSSTLEINS | is tethered to the | |
| | | SGQLTVRSTSPNLRYPIADVSGGIGMSPNYR | C-terminal pσ1 | |
| | | FRQSMWIGIVSYSGSGLNWRVQVNSDIFIVD | domain by a GRPG | |
| | | DYIHICLPAFDGFSIADGGDLSLNFVTGLLPP | linker (italics- | |
| | | LLTGDTEPAFHNDVVTYGAQTVAIGLSSGG | boxed) | |
| | | APQYMSKNLWVEQWQDGVLRLRVEGGGSI | | |
| | | THSNSKWPAMTVSYPRSFT | | |

*In some embodiments, the fusion protein represented by SEQ ID NO: 5 is expressed with additional sequence, where the bracketed MG is replaced by the following: MRGSHHHHHHGMASMTGGQQMGRDLYDDDDKDHPFTEFGAGGVGGTP (SEQ ID NO: 13). This additional sequence corresponds to the initial Methionine, His and either protein tags, additional amino acids from the subcloning strategy, and an additional cathepsin S, B site (underlined).
**In some embodiments, the fusion protein represented by SEQ ID NO: 6 is expressed with additional sequence, where the following is inserted prior to the bracketed M: MRGSHHHHHHGMASMTGGQQMGRDLYDDDDKDHPFTEFLEGAGGVGGTP (SEQ ID NO: 14). This additional sequence corresponds to the initial Methionine, His and either protein tags, additional amino acids from the subcloning strategy, and an additional cathepsin S, B site (underlined).

It will be appreciated that the representative antigenic polypeptides and their sources are non-limiting examples and that any known antigenic polypeptide for which tolerance is desired is encompassed by the present disclosure. Further, as described above, the particular antigenic polypeptide or polypeptides incorporated into the disclosed fusion protein need not be the full length polypeptide from the protein source, but instead may be “derived therefrom”. In some embodiments, the polypeptide is a subcomponent, such as a fragment or fusion of multiple fragments, of the full-length source protein. The incorporation of such derivatives can be advantageous for purposes of production of the fusion protein. In this regard, recombinant expression of the fusion protein can be more efficient for smaller overall proteins, or can be enhanced with the exclusion of particularly problematic domains of the source protein. Furthermore, in some instances the resulting fusion protein will be more effective at inducing tolerance because the fusion protein contains the one or more critical antigens/epitopes while excluding other domains that may diminish the tolerization effect.

In instances where the intended source protein is not incorporated in its entirety, but rather fragments thereof are used as the antigenic polypeptide in the fusion protein, the selection of fragments as the polypeptide can be made based on various parameters. For instance, the polypeptide preferably comprises an MHC Class I and/or MHC Class II epitope (also referred to as a T cell epitope). Such epitopes are short, linear lengths of polypeptides that MEW molecules can process and present to T cells. Cells in the mucosa, such as in the GALT and the NALT regions, express both MHC Class I and II, and can play a role in tolerization to antigens. Epitopes presented by MEW class I molecules are typically peptides between 8 and 11 amino acids in length, whereas MHC class II molecules present longer peptides, 13-17 amino acids in length. Accordingly, the antigenic polypeptide will typically comprise at least 8 amino acids. However, it will be appreciated that the polypeptide can be much larger, limited only by the ability of the expression or synthesis system to produce the final fusion protein. Specific MEW epitopes can be readily predicted from the selected source protein sequence. As indicated, the lengths of the typical MHC epitopes are known. Furthermore, MHC Class I and MEW Class II epitopes have characteristic anchor points that rely on generalized sequence patterns. Thus, algorithms exist to predict the MHC epitopes from a source sequence. See, e.g., Koren, E., et al., “Clinical validation of the “in silico” prediction of immunogenicity of a human recombinant therapeutic protein,” Clinical Immunol. 124:26-32 (2007), incorporated herein by reference. Many useful applications are available on the world wide web to apply various prediction algorithms to provided source sequences. For example, the Immune Epitope Database (IEDB) and Analysis Resource provides a website at the address iedb.org, which funded by a contract from the National Institute of Allergy and Infectious Diseases. This resource offers easy searching of experimental database with data characterizing known T cell epitopes (presented via MHC) as studied in humans, non-human primates, and other animal species. Epitopes involved in allergy, autoimmunity, and transplant are included. This resource also hosts tools to assist in the prediction and analysis of B cell and T cell epitopes. With the application of such an algorithm to any of source protein sequence, such as the illustrative source proteins described above, a person of ordinary skill in the art can readily select the best epitope(s) to include in the one or more polypeptide(s) that is ultimately incorporated into the fusion protein.

As an example, Arah2 peptides containing dominant CD4+ T cell epitopes are known in the art. See for example, Prickett, S. R., et al., “Arah2 Peptides Containing Dominant CD4+ T-cell Epitopes: Candidates for a Peanut Allergy Therapeutic,” J. Allergy Clin. Immunol. 127:608-615 (2011) and Glasploe, I. N., et al., “Characterization of the T-cell Epitopes of a Major Peanut Allergen, Ara h 2,” Allergy 60:35-40 (2005), incorporated herein in their entirety. Prickett et al. disclose five dominant CD4+ T-cell epitopes including aa32-44 (SQLERANLRPCEQ; SEQ ID NO:8), aa37-47 (ANLRPCEQHLM; SEQ ID NO:9), aa91-102 (ELNEFENNQRCM; SEQ ID NO:10), aa95-107 (FENNQRCM; SEQ ID NO:11), and aa128-141 (RELRNLPQQCGLRA, SEQ ID NO:12). In combination, these epitopes were presented by HLA-DR, HLA-DP and HLA-DQ molecules and recognized by T cells from all of the subjects tested. Any fusion polypeptide of the present disclosure would include at least one and likely more than one T cell epitope.

T cell peptide epitopes are also known for α-gliadin and include, for example, and not by limitation, a 33 amino acid sequence comprising aa56-88 to contain six partly overlapping copes of three DQ2-restricted T cell epitopes. See, for example, Shan, L., et al., “Structural Basis for Gluten Intolerance in Celiac Sprue,” Science 297:2275-2279 (2002) and Qiao, S. W., et al., “Antigen Presentation to Celiac Lesion-Derived T Cells of a 33-mer Gliadin Peptide Naturally Formed by Gastrointestinal Digestion,” J. Immunol. 173:1757-1759 (2004).

The fusion protein can also include various tags that can assist the expression, production, or later analysis (e.g., visualization) thereof. Such tags are well-known and are commonly used in the art during the production of recombinant fusion proteins. Tags can be attached at the N- or C-terminus of the antigen construct but are usually placed at the N-terminal end. Examples of tags are: NusA, thioredoxin, maltose binding protein, small ubiquitin-like molecules (Sumo-tag), and His-repeats. If desired, to facilitate removal of the tag during purification, a unique protease site can be inserted between the tag and the fusion protein per se. Such protease sites may include those for thrombin, factor Xa, enterokinase, PreScission™, Sumo™. Alternatively, removal of the tag may be achieved via inclusion of an intein sequence between the tag and the fusion protein per se. Inteins are self-cleaving proteins and in response to a stimulus (e.g., lowered pH) are capable of self-splicing at the junction between the intein and the antigen construct, thus eliminating the need for the addition of specific proteases. Examples of inteins include domains derived from Mycobacterium tuberculosis (RecA), and Pyrococcus horikoshii (RadA) (Fong, et al., Trends Biotechnol. 28:272-279 (2010)).

To facilitate purification, the fusion protein can include one or more purification tags to enable specific chromatography steps (e.g., metal ion chelating, affinity chromatography) to be included in the purification processes. Such purification tags can, for example, include: repeat histidine residues (e.g., 6-10 histidine residues), maltose binding protein, glutathione S-transferase; and streptavidin. These tags can be attached at the N- and/or C-terminus of the polypeptide antigens of the invention. To facilitate removal of such tags during purification, protease sites and/or inteins (examples above) can be inserted between the polypeptide and the purification tag(s).

The fusion protein can also include a visualization tag. For example, this tag can include portions of proteins that are known to provide a detectable signal, such as fluorescence. Alternatively, any tag herein can provide an epitope for specific recognition and binding by a detectably labeled antibody or antibody fragment, or any other molecule capable of emitting detectable light or energy. Exemplary tags that can provide a detectable signal include GFP, any of the numerous related GFP variants known in the art to similarly fluoresce upon stimulation, such as blue fluorescent protein, cyan fluorescent protein, and yellow fluorescent protein, mCherry, and the like. The visualization tag can also serve as an epitope for binding and isolation of the fusion protein.

In another aspect, the present disclosure provides a pharmaceutical composition comprising the isolated fusion protein described herein. The pharmaceutical composition can also comprise pharmaceutically acceptable carriers, stabilizers, excipients, and other additives to provide an appropriate formulation for the preferred route of administration, as is familiar in the art. Generally, oral and intranasal routes of administration are addressed herein, but other known routes of administration are contemplated as well. An exemplary formulation for intranasal administration can include components to facilitate inhalation and delivery to the mucosal surface. For example, such formulations can include aerosols, particulates, and the like. In general, the goal for particle size for inhalation is about 1 μm or less. Such formulation can be delivered by in the form of an aerosol spray. Oral formulations may be liquid (for example, syrups, solutions, or suspensions), or solid (for example, powders, pills, tablets, or capsules). For solid compositions, conventional non-toxic solid carriers can include pharmaceutical grades of mannitol, lactose, starch, or magnesium stearate. Actual methods of preparing such dosage forms are known, or will be apparent, to those of ordinary skill in the art. Solid formulations for oral administration can also comprise known binding agents, fillers, lubricants, disintegrants, or wetting agents. The dose form can also be coated. Liquids for oral administration can contain additional additives such as suspending agents, emulsifiers, non-aqueous vehicles, and preservatives.

In another aspect, the disclosure provides a nucleic acid encoding the isolated fusion protein described herein.

As used herein, the term “nucleic acid” refers to any polymer molecule that comprises multiple nucleotide subunits (i.e., a polynucleotide). Nucleic acids encompassed by the present disclosure can include deoxyribonucleotide polymer (DNA), ribonucleotide polymer (RNA), cDNA or a synthetic nucleic acid known in the art.

Nucleotide subunits of the nucleic acid polymers can be naturally occurring or artificial or modified. A nucleotide typically contains a nucleobase, a sugar, and at least one phosphate group. The nucleobase is typically heterocyclic. Canonical nucleobases include purines and pyrimidines and more specifically adenine (A), guanine (G), thymine (T) (or typically in RNA, uracil (U) instead of thymine (T)), and cytosine (C)). The sugar is typically a pentose sugar. Suitable sugars include, but are not limited to, ribose and deoxyribose. The nucleotide is typically a ribonucleotide or deoxyribonucleotide. The nucleotide typically contains a monophosphate, diphosphate, or triphosphate. These are generally referred to herein as nucleotides or nucleotide residues to indicate the subunit. Without specific identification, the general terms nucleotides, nucleotide residues, and the like, are not intended to imply any specific structure or identity. The nucleotides can also be synthetic or modified.

In another aspect, the disclosure provides vectors comprising the nucleic acid sequences described herein, such as a vector comprising a nucleic acid sequence encoding the polypeptide described above. Such vectors are useful for the recombinant expression of the fusion protein in a cell-based expression system. Such expression systems are well-known in the art, and include cell strains optimized for recombinant expression of genes associated with specific vectors parameters. For example, any vector described herein can further comprise a promoter sequence to facilitate expression of the nucleic acid encoding the fusion protein in the intended cellular expression system. Any appropriate promoter can be used, such as a constitutive promoter or inducible promoter, appropriate for the expression system to be used, as known in the art. For example, an inducible promoter can comprise an acetamide-inducible promoter. Additionally, the vector can also include selectable markers, such as antibiotic or toxin resistance genes, that will confer protection against such applied agents. In this manner, cells that are successfully transformed with the operational vector can be retained in culture and the non-transformed cells in the system can be removed.

Also provided are cultured cells transfected with any vector described herein, or progeny thereof, wherein the cell is capable of expressing a fusion protein, as described above. The cell can be prokaryotic, such as E. coli, or eukaryotic, such as insect or mammalian.

In another aspect, the present disclosure provides a method for inducing tolerance to a protein, such as an allergen polypeptide, an autoantigen, or a biological therapeutic. The method comprises administering a pharmaceutically effective amount of the isolated fusion protein or the pharmaceutical composition, as described herein, to a subject in need thereof. The fusion protein comprises a polypeptide derived from the protein to which tolerance is desired. Therefore, the fusion protein need not necessarily comprise the entire protein. It is preferable, however, that the fusion protein, and specifically the antigenic polypeptide, comprises the most reactive epitopes of the protein to induce a more comprehensive tolerance to the source protein.

In some embodiments, the method consists of administering a single dose of the effective amount of the isolated fusion polypeptide. In other embodiments, the method can further comprise a second, third, fourth, or more additional administrations. In embodiments with multiple administrations, each administration need not contain the same dose. Furthermore, in some embodiments, each administration need not contain the same fusion protein, but can contain additional or different antigenic polypeptide(s).

Illustrative, non-limiting effective doses of isolated fusion polypeptide include less than about 100 mg, 75 mg, 50 mg, 25 mg, 20 mg, 15 mg, 10 mg, 9 mg, 8 mg, 7 mg, 6 mg, 5 mg, 4 mg, 3 mg, 2 mg, 1.5 mg, 1 mg, 750 μg, 500 μg, 250 μg, 100 μg, 75 μg, 50 μg, or 25 μg, or any number or range therein.

In another aspect, the disclosure provides a method for screening a subject to provide a personalized fusion protein to maximize the tolerization to an allergenic polypeptide, autoantigen, or biological therapeutic, by the individual. The method includes obtaining peripheral blood mononuclear cells (PBMCs) from the subject. This can involve affirmatively obtaining a blood sample and isolating the PBMCs. The isolated PBMCs are contacted with an isolated candidate antigen, either whole or a substantial fragment (portion) thereof. The PBMCs are monitored for T cell proliferation. In some embodiments, PBMC fractions can be exposed separately to a panel of candidate allergens/antigens, or a panel of different fragments of one or more candidate allergens/antigens. The antigen/allergen, or fragment thereof, that elicits a strong proliferation of T cells in the proliferation assay is chosen for inclusion in the fusion protein to be administered to the subject from whom the PBMCs were obtained. As an example, a patient with multiple sclerosis (MS) can be tested for an appropriate therapeutic fusion protein. PBMCs can be exposed to myelin basic protein and myelin oligodendrocyte glycolprotein (MOG), fragments thereof, various fusions of fragments thereof, or any other known antigen that is suspected to contribute to MS. The antigens that elicit the greatest T cell proliferation can be incorporated into a therapeutic fusion protein, as described herein, for an enhanced treatment personalized to the unique characteristics of the patient's own PBMC population. As another example, PBMCs from a patient suffering from a peanut allergy can be exposed to various known proteins from peanut, fragments thereof, or fusions of various fragments thereof. The reactivity of the PBMCs against the panel peanut allergens can be monitored in a T cell proliferation assay, and only the antigen polypeptide(s) eliciting a high reactivity with the PBMCs can be incorporated into one or more fusion protein constructs, as described herein. Accordingly, the patient will only receive one or more fusion protein constructs incorporating the most highly reactive allergen polypeptides for that subject.

It is noted that, as used herein, the use of the term “or” in the claims means “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

The practice of the present disclosure employs, unless otherwise indicated, conventional immunological and molecular biological techniques and pharmacology within the skill of the art. Such techniques are well-known to the skilled worker, and are explained fully in the literature. See, e.g., Coligan, Dunn, Ploegh, Speicher and Wingfield “Current Protocols in Protein Science” (1999), Volume I and II (John Wiley & Sons Inc.); Sambrook et al., “Molecular Cloning: A Laboratory Manual” (1989), 2nd Edition (Cold Spring Harbor Laboratory Press); and Prescott, Harley and Klein “Microbiology” (1999), 4^{th} Edition (WBC McGraw-Hill). Additionally, such considerations as routes of administration, antigen dose, number, frequency of administrations, and appropriate formulations are all matters of optimization within the scope of the ordinary skill in the art.

All publications, patents, and patent applications cited herein, whether supra or infra, are hereby incorporated by reference in their entirety. However, publications mentioned herein are cited for the purpose of describing and disclosing the protocols, reagents, and the like, which are reported in the publications and which might be used in connection with the invention.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application.

Disclosed are materials, compositions, and components that can be used for, in conjunction with, in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. Additionally, it is understood that the embodiments described herein can be implemented using any suitable material such as those described elsewhere herein or as known in the art.

The following examples provide illustrative, non-limiting descriptions of experimental approaches for creating, testing, and using the disclosed tolerance therapeutic.

### Example 1

Introduction: This section describes an exemplary approach for producing a fusion protein with a cleavable linker that can induce tolerance to polypeptide antigen, such as an endogenous autoantigen (e.g., pro-insulin antigen, multiple sclerosis antigen) or exogenous allergen (e.g., peanut allergen, gluten).

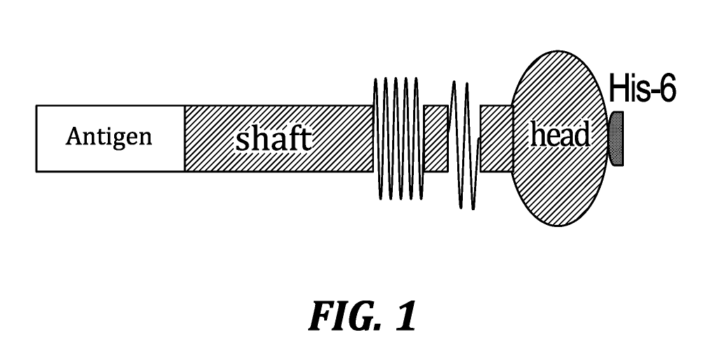

Experimental Design: A cDNA encoding fusion polypeptide, which incorporates one or more antigen(s)/allergen(s) with a targeting domain, can be synthesized with appropriate restriction sites and cloned into a suitable expression vector generating the polypeptide-pσ1 fusion protein. See Table 1 for exemplary constructs that incorporate gluten epitopes (from α-gliadin, ω-gliadin, Hordein, and Secalin) to address celiac disease, a combination of epitopes of autoantigens (from MOG, MBP, and PLP antigens) to address multiple sclerosis, or multiple epitopes from pro-insulin to address type I diabetes. It will be understood that this format can be applied to any protein allergen(s), autoantigen(s), or biological therapeutic(s) of interest with one or more polypeptide epitopes from the protein of interest. For example, epitopes from the Arah2 (or other) peanut allergen can be incorporated along with cleavable linkers. The construct generated for study can a poly-histidine tag for affinity purification (see, e.g., FIG. 1), although the His-tag can be omitted, for example, for therapeutic uses. After initial characterization, expression can be scaled up and material can be purified and characterized by SDS-PAGE and western blot using antibodies to pσ1 and the incorporated antigen/epitope. Functional activity of pσ1 fusion protein can be demonstrated in vitro using both Hela and L-cell binding assays. An exemplary target production level is at least 10 mg of purified protein for experimental characterization and assays.

1) Vector Construction and Expression: A “VTC-GT1” DNA sequence containing 7 gluten immuno-dominant epitopes can be commercially synthesized (GeneArt, Thermo Fisher Scientific) and subcloned into an Escherichia coli expression vector (see, e.g., SEQ ID NO:5 and line 1 of Table 1 for an exemplary sequence of such a construct). The 7 dominant T cell epitopes can include the DQ2 epitopes previously identified from α-gliadin, ω-gliadin, hordein, and secalin as being highly antigenic across celiac patients (see Tye-Din, J. A., et al., “Comprehensive, quantitative mapping of T cell epitopes in gluten in celiac disease.” Sci Transl Med 2:41 (2010); Shan, L. et al., “Structural basis for gluten intolerance in celiac sprue.” Science 297:2275-9 (2002); Qiao, S. W., et al., “Antigen presentation to celiac lesion-derived T cells of a 33-mer gliadin peptide naturally formed by gastrointestinal digestion.” J Immunol 173:1757-62 (2004). Selection of polypeptides from various proteins found in gluten can be selected based on the presence of circulating T cells in humans that are activated by those polypeptides. The complete sequence encoding gluten polypeptide epitopes with the cleavable linkers separating the polypeptide epitopes can be fused to pσ1, codon optimized for E. coli expression, and the DNA subcloned into any bacterial expression vector including pET system vectors such as the pET100 vector (Thermo Fisher Scientific) utilized for protein studies. The sub-cloning places the expression of the fusion protein under control of the T7 promotor with a lac operator (lacO) allowing induction by Isopropyl β-D-1-thiogalactopyranoside (IPTG). In addition, the vector incorporates an amino terminal poly histidine tag for VTC-GT1 protein production, purification, and characterization. pET100:VTC-GT1 was transformed into chemically competent BL21(DE3) (Thermo Fisher Scientific). Carbenicillin was used to select for BL21(DE3) harboring the pET100 vector. Ampicillin could be used to select for pET100, and additional expression organisms and suitable expression vectors could potentially be utilized as well.

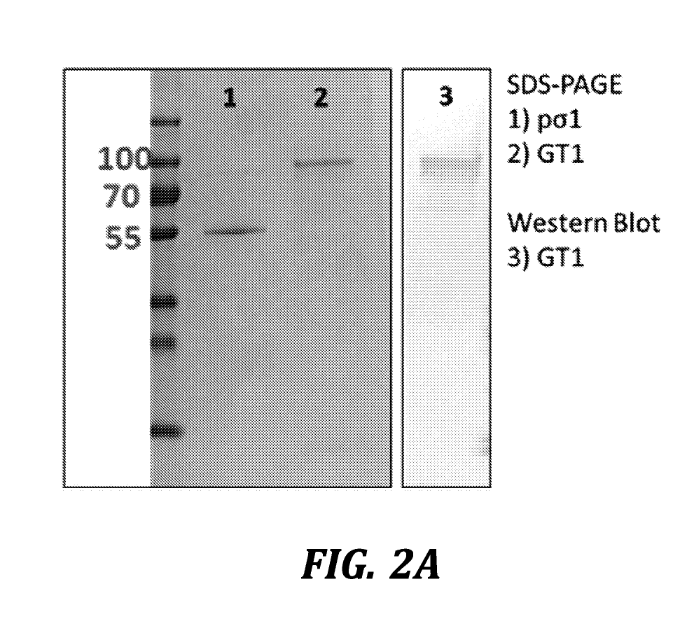

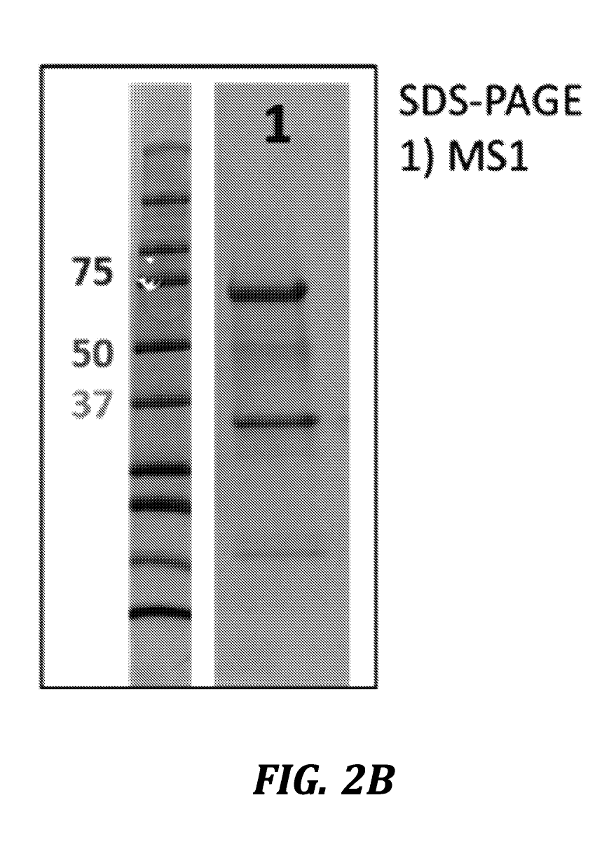

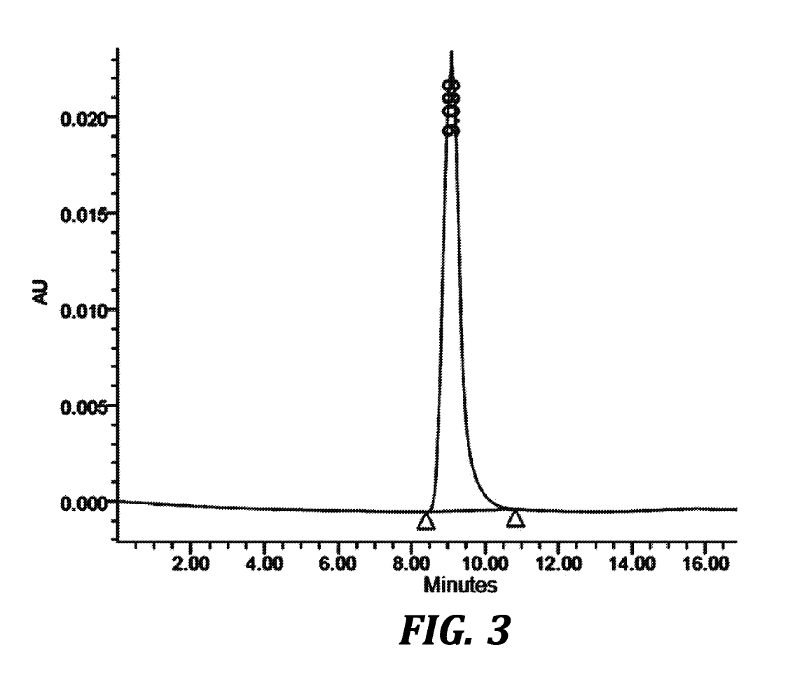

2) Purification and Characterization: Recombinant proteins can be extracted from bacterial cells using lysozyme and a sonicator, and purified on a Talon metal affinity resin (BD Biosciences), according to manufacturer's instructions. Nickel affinity chromatography can be used for protein purification as well. Proteins can be assessed for purity and quality by Coomassie-stained polyacrylamide gels and by Western blot analysis using a polyclonal rabbit anti-pσ1 or antibodies that specifically bind to the pσ1 region and/or poly-his. All recombinant proteins should migrate as a single band with the expected molecular weight. Additionally, VTC-GT1 can be expressed and purified from E. coli inclusion bodies using standard inclusion body isolation and purification protocols (see Palmer, I. and Wingfield, P. T., “Preparation and Extraction of Insoluble (Inclusion-Body) Proteins from Eschericia coli,” Curr. Protoc. Protein Sci. Chapter: Unit-6.3 (2004)). BL21(DE3) pET100:VTC-GT1 was grown in 2×YT media, supplemented with carbenicillin at 50 ug/mL. Any rich media, e.g. LB, could potentially be used as well. After reaching an OD600 of 0.6, protein expression was induced for 4 hours with the addition of IPTG at 1 mM final. Cell pellets were harvested and frozen at −80 C until protein purification the next day. To purify VTC-GT1, cell pellets were thawed on ice, and resuspended in cell lysis buffer (100 mM NaH2PO4, 10 mM Tris-Cl, 1 mg/mL lysozyme, 1×HALT protease inhibitor cocktail (Thermo Scientific), 5.5 mM 2-Mercaptoethanol, pH 8). Following a 30 minute incubation on ice, cells were sonicated at 25% duty six times for 30 sec on ice, with a 30 sec cooling on ice. Whole cell extracts were spun at 12 k rpm for 60 min, and the supernatant was discarded. Insoluble pellets were washed three times in wash buffer (100 mM NaH2PO4, 10 mM Tris-Cl, 2 M urea, 5.5 mM 2-Mercaptoethanol, pH 8) supplemented with Triton X-100 at 5%, followed by two washes in wash buffer without Triton X-100. Samples were spun at 12 k rpm for 30 min after each wash. Additional washes can be utilized until the discarded supernatant is clear. VTC-GT1 was extracted in extraction buffer (100 mM NaH2PO4, 10 mM Tris-Cl, 8 M urea, 5.5 mM 2-Mercaptoethanol, pH 8), and dialyzed against 1×PBS. VTC-GT1 was isolated at greater than 95% purity as analyzed by Coomassie stained polyacrylamide gel and size exclusion chromatography (FIGS. 2-3).

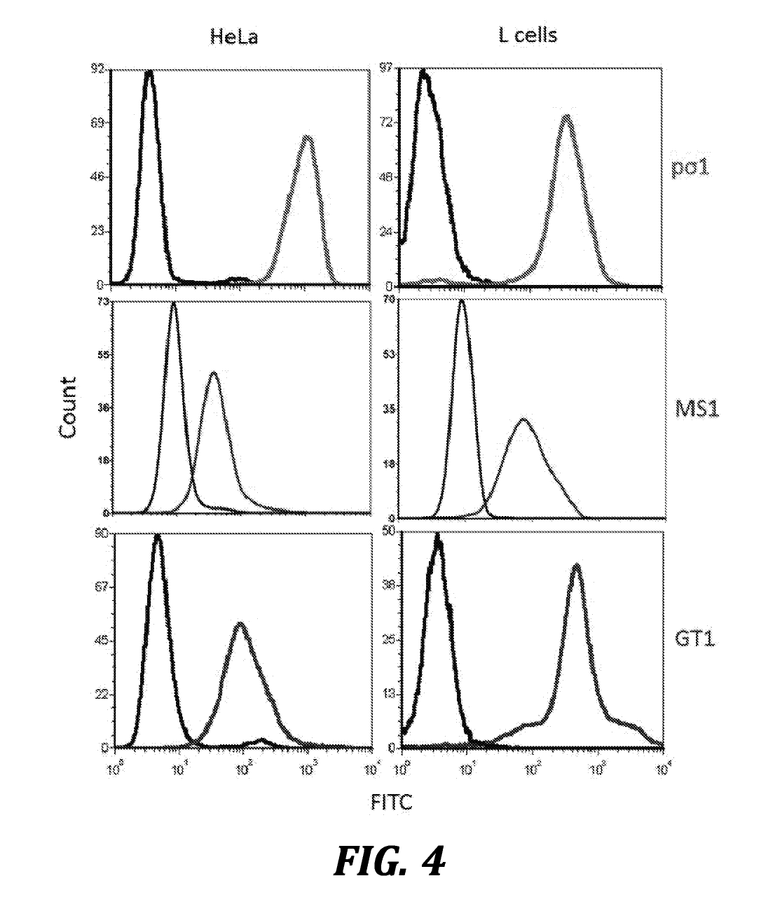

3) Cell Binding Activity: pσ1 has been shown to interact with at least two host receptors via separate binding domains. The head domain binds with a component of tight junctions expressed by L-cells, whereas sequences contained within the fibrous tail domain bind terminal α-linked sialic acid residues on host cells, including HeLa cells (see, e.g., Guglielmi, K. M., et al., “Attachment and cell entry of mammalian orthoreovirus,” Curr. Top. Microbiol. Immunol. 309:1-38 (2006); Turner, D. L., et al., “Site-directed Mutagenesis of the C-terminal Portion of Reovirus Protein Sigma 1: Evidence for a Conformation-dependent Receptor Binding Domain,” Virology 186:219-227 (1992); Nibert, M. L., et al., “Infectious Subvirion Particles of Reovirus Type 3 Dearing Exhibit a Loss in Infectivity and Contain a Cleaved Sigma 1 Protein,” J. Virol. 69:5057-5067 (1995); and Lee, P. W. and Leone, G., “Reovirus Protein Sigma 1: From Cell Attachment to Protein Oligomerization and Folding Mechanisms,” Bioessays 16:199-206 (1994); and Barton, E. S., et al., “Utilization of Sialic Acid as a Coreceptor Enhances Reovirus Attachment by Multistep Adhesion Strengthening,” J. Biol. Chem. 276:2200-2211 (2000), each of which is incorporated by reference in its entirety. VTC-GT1 cell binding can be measured by FACS analysis. Washed HeLa or L-cells (3×10⁴ cells) can be incubated with or without 10-50 μg pσ1 fusion protein or pσ1 (without fused antigenic polypeptide(s)) for 30 minutes on ice. Following a wash, rabbit polyclonal anti-pσ1 or commercially prepared polyclonal Ab (against the antigen polypeptide(s) or isotype control Ab can be incubated for 30 minutes on ice. Following wash, FITC-labeled goat-anti-rabbit IgG (Jackson ImmunoResearch Laboratories) can be incubated for 30 minutes on ice. Following wash, cells can be read on flow cytometry to measure extent of bound pσ1 fusion protein. VTC-GT1 is soluble in PBS and binds HeLa and L-cells with similar affinity as MOG-pσ1 and pσ1 proteins, suggesting that the head and tail/shaft domains of pσ1 in VTC-GT1 are functional (FIG. 4).

As an alternative strategy, a tripartite cloning strategy can be applied to pPICZ (Invitrogen®) expression vector for use in the yeast Pichia pastoris. In our experience, having a choice between E. coli and P. pastoris significantly increases the chance of successful expressing greater than 10 mg of the recombinant protein.

Expected results: It is anticipated that the above strategy can produce over 10 mg of purified fusion protein, such as illustrated in Table 1, with >98% purity with binding activity to L and HELA cells comparable to pσ1.

### Example 2

Introduction: This section describes an exemplary approach for determining the optimal oral dose of a fusion protein produced as described in Example 1. This is described in the context of fusion proteins that incorporate gluten polypeptides with the pσ1 targeting polypeptide, although it will be understood that the protocol can be readily modified to address dose optimization of any fusion protein described herein.

Experimental Design: Determination of the optimal oral dose of a pσ1 fusion protein comprising the gluten polypeptides is described. The study can also include control animals that are dosed with the individual fusion components, gluten polypeptides and pσ1, to demonstrate that such proteins do not generate efficacy at the highest dose of VTC-GT1 fusion protein used.

Determination of the optimal oral dose of the VTC-GT1 fusion protein: Groups of 5 mice can be treated orally with either PBS or increasing doses of the VTC-GT1 fusion protein at 10, 50, 100, and 500 μg per mouse. Seven days later, mice can be orally immunized with 1 mg of a polypeptide containing the same gluten epitopes found in VTC-GT1 plus 15 μg of cholera toxin at least three times at weekly intervals. (see Kato H., et al., “Oral tolerance revisited: prior oral tolerization abrogates cholera toxin-induced mucosal IgA responses.” J. Immunol 166:3114-3121 (2001). Several characteristics can be observed, such as the number of FoxP3+ T cells, T cell responses and cytokine responses.

Number of FoxP3+ T cells: The number of CD4+CD25+FoxP3+, and CD4+CD25+Foxp3− T cells present in the spleen, HNLN, MLNs, and PPs can be evaluated using flow cytometric analysis. CD4+ T cells from spleen, HNLN, MLNs, and PPs can purified by use of an automated magnetic activated cell sorter (AutoMACS) system (Miltenyi Biotec, Auburn, Calif.). The purified CD4+ T cell fraction can then be suspended in complete RPMI 1640 (4×10⁶ cells/ml) and incubated with labeled monoclonal antibodies to CD25 and FoxP3. The cells numbers can then be calculated from the flow cytometric analysis to determine the number of Foxp3+ cells.

T cell Responses: CD4+ T cells from spleen, MLNs, and PPs can purified by use of an automated magnetic activated cell sorter (AutoMACS) system (Miltenyi Biotec, Auburn, Calif.). The purified CD4+ T cell fraction can then be suspended in complete RPMI 1640 (4×106 cells/ml) and cultured with or without one mg/ml of the gluten antigenic polypeptides in the presence of T cell-depleted, irradiated (3000 rad) splenic antigen-presenting cells (APCs) taken from non-immunized mice for five days. To assess polypeptide-specific T cell proliferative responses, an aliquot of 0.5 μCi of tritiated [3H]-TdR (Amersham Biosciences, Arlington Heights, Ill.) can be added during the final 18 hr of incubation, and the amount of [3H]-TdR incorporation can be determined by scintillation counting. The supernatants of identically treated T cell cultures not incubated with [3H]-TdR can then subjected to a cytokine-specific ELISA as described below.

T cell Cytokine Production: The cytokines and levels produced by CD4+ T cells restimulated in vitro with the gluten polypetides can be measured by an ELISA assay. The ELISA for measuring IFN-γ, IL-2, IL-4, IL-5, IL-6, IL-10, IL-17, IL-28, and TGF-β1 can be purchased from various vendors. The levels of Ag-specific cytokine production can be calculated by subtracting the results of control cultures (e.g., without Ag stimulation) from those of Ag-stimulated cultures.

Expected Outcome: It is expected that the optimal effective dose of VTC-GT1 fusion protein will be 50 μg. It is also anticipated that significant induction of FoxP3+ T cells will be measured and tolerance to the gluten polypeptides will be observed across the following parameters evaluated: CD4 T cell responses and anti-inflammatory cytokine production, which includes IL-4, IL-10 and IL-28.

Alternative Strategies: If needed, the sensitization protocol described above can be adjusted by varying amount and location of vaccination to the gluten polypeptides, as well as the type of adjuvant. In addition, the number of VTC-GT1 fusion protein treatments at the planned doses can be extended to determine the optimal dose.

### Example 3

Introduction: This section describes an exemplary approach for producing a fusion protein with a cleavable linker that can induce tolerance to polypeptide antigen, such as an endogenous autoantigen (e.g., pro-insulin antigen) or exogenous allergen (e.g., peanut allergen).

Experimental Design: A cDNA encoding fusion polypeptide, which incorporates one or more antigen(s)/allergen(s) with a targeting domain, can be synthesized with appropriate restriction sites and cloned into a suitable expression vector generating the polypeptide-pσ1 fusion protein. See Table 1 for exemplary constructs that incorporate gluten epitopes (from α-gliadin, ω-gliadin, Hordein, and Secalin) to address celiac disease, a combination of epitopes of autoantigens (from MOG, MBP, and PLP antigens) to address multiple sclerosis, or multiple eptiopes from pro-insulin to address type I diabetes. It will be understood that this format can be applied to any protein allergen(s), autoantigen(s), or biological therapeutic(s) of interest with one or more polypeptide epitopes from the protein of interest. For example, epitopes from the Arah2 (or other) peanut allergen can be incorporated along with cleavable linkers. The construct generated for study can a poly-histidine tag for affinity purification (see, e.g., FIG. 1), although the His-tag can be omitted, for example, for therapeutic uses. After initial characterization, expression can be scaled up and material can be purified and characterized by SDS-PAGE and western blot using antibodies to pσ1 and the incorporated antigen/epitope. Functional activity of pσ1 fusion protein can be demonstrated in vitro using both Hela and L-cell binding assays. An exemplary target production level is at least 10 mg of purified protein for experimental characterization and assays.

1) Vector Construction and Expression: A “VTC-MS1” DNA sequence containing 6 auto-antigenic epitopes can be commercially synthesized (GeneArt, Thermo Fisher Scientific) and subcloned into an Escherichia coli expression vector (see, e.g., SEQ ID NO:6 and line 2 of Table 1 for an exemplary sequence of such a construct). The 6 dominant T cell epitopes can include the MBP, PLP, and MOG epitopes that have been identified in MS patients that have circulating auto-reactive T cells and antibodies specific to the MBP, PLP, and MOG epitopes (see Riedhammer C, Weissert R. Antigen Presentation, Autoantigens, and Immune Regulation in Multiple Sclerosis and Other Autoimmune Diseases. Front Immunol. 2015; 6:322.; Kerlero de Rosbo N, Milo R, Lees M B, Burger D, Bernard C C, Ben-Nun A. Reactivity to myelin antigens in multiple sclerosis. Peripheral blood lymphocytes respond predominantly to myelin oligodendrocyte glycoprotein. J Clin Invest. 1993; 92(6):2602-2608.; Quintana F J, Farez M F, Izquierdo G, Lucas M, Cohen I R, Weiner H L. Antigen microarrays identify CNS-produced autoantibodies in RRMS. Neurology. 2012; 78(8):532-539.; Zhang J, Markovic-Plese S, Lacet B, Raus J, Weiner H L, Hafler D A. Increased frequency of interleukin 2-responsive T cells specific for myelin basic protein and proteolipid protein in peripheral blood and cerebrospinal fluid of patients with multiple sclerosis. J Exp Med. 1994; 179(3):973-984.). Selection of polypeptides from various myelin proteins can be selected based on the presence of circulating T cells in humans that are activated by those polypeptides. The complete sequence encoding auto-antigenic MS polypeptide epitopes with the cleavable linkers separating the polypeptide epitopes can be fused to pσ1, codon optimized for E. coli expression, and the DNA subcloned into any bacterial expression vector including pET system vectors such as the pET100 vector (Thermo Fisher Scientific) utilized for protein studies. Additional expression organisms and suitable expression vectors could potentially be utilized as well. The sub-cloning places the expression of the fusion protein under control of the T7 promotor with a lac operator (lacO) allowing induction by Isopropyl β-D-1-thiogalactopyranoside (IPTG). In addition, the vector incorporates an amino terminal poly histidine tag. For VTC-MS1 protein production, purification, and characterization, pET100:VTC-MS1 was transformed into chemically competent BL21(DE3) (Thermo Fisher Scientific). Carbenicillin was used to select for BL21(DE3) harboring the pET100 vector. Ampicillin could be used to select for pET100, and additional expression organisms and suitable expression vectors could potentially be utilized as well.

2) Purification and Characterization: Recombinant proteins can be extracted from bacterial cells using lysozyme and a sonicator, and purified on a Talon metal affinity resin (BD Biosciences), according to manufacturer's instructions. Nickel affinity chromatography can be used for protein purification as well. Proteins can be assessed for purity and quality by Coomassie-stained polyacrylamide gels and by Western blot analysis using a polyclonal rabbit anti-pσ1 or antibodies that specifically bind to the pσ1 and poly-his. All recombinant proteins should migrate as a single band with the expected molecular weight. Additionally, VTC-MS1 can be expressed and purified from E. coli inclusion bodies using standard inclusion body isolation and purification protocols (see Palmer, I. and Wingfield, P. T., “Preparation and Extraction of Insoluble (Inclusion-Body) Proteins from Eschericia coli,” Curr. Protoc. Protein Sci. Chapter: Unit-6.3 (2004)). BL21(DE3) pET100:VTC-MS1 was grown in 2×YT media, supplemented with carbenicillin at 50 ug/mL. Any rich media, e.g. LB, could potentially be used as well. After reaching an OD600 of 0.6, protein expression was induced for 4 hours with the addition of IPTG at 1 mM final. Cell pellets were harvested and frozen at −80 C until protein purification the next day. To purify VTC-MS1, cell pellets were thawed on ice, and resuspended in cell lysis buffer (100 mM NaH2PO4, 10 mM Tris-Cl, 1 mg/mL lysozyme, 1×HALT protease inhibitor cocktail (Thermo Scientific), 5.5 mM 2-Mercaptoethanol, pH 8). Following a 30 minute incubation on ice, cells were sonicated at 25% duty six times for 30 sec on ice, with a 30 sec cooling on ice. Whole cell extracts were spun at 12 k rpm for 60 min, and the supernatant was discarded. Insoluble pellets were washed three times in wash buffer (100 mM NaH2PO4, 10 mM Tris-Cl, 2 M urea, 5.5 mM 2-Mercaptoethanol, pH 8) supplemented with Triton X-100 at 5%, followed by two washes in wash buffer without Triton X-100. Samples were spun at 12 k rpm for 30 min after each wash. Additional washes can be utilized until the discarded supernatant is clear. VTC-MS1 was extracted in extraction buffer (100 mM NaH2PO4, 10 mM Tris-Cl, 8 M urea, 5.5 mM 2-Mercaptoethanol, pH 8). VTC-MS1 was expressed and isolated from BL21(DE3) as analyzed by Coomassie stained polyacrylamide gel (FIG. 2)

3) Cell Binding Activity: pσ1 has been shown to interact with at least two host receptors via separate binding domains. The head domain binds with a component of tight junctions expressed by L-cells, whereas sequences contained within the fibrous tail domain bind terminal α-linked sialic acid residues on host cells, including HeLa cells (see, e.g., Guglielmi, K. M., et al., “Attachment and cell entry of mammalian orthoreovirus,” Curr. Top. Microbiol. Immunol. 309:1-38 (2006); Turner, D. L., et al., “Site-directed Mutagenesis of the C-terminal Portion of Reovirus Protein Sigma 1: Evidence for a Conformation-dependent Receptor Binding Domain,” Virology 186:219-227 (1992); Nibert, M. L., et al., “Infectious Subvirion Particles of Reovirus Type 3 Dearing Exhibit a Loss in Infectivity and Contain a Cleaved Sigma 1 Protein,” J. Virol. 69:5057-5067 (1995); and Lee, P. W. and Leone, G., “Reovirus Protein Sigma 1: From Cell Attachment to Protein Oligomerization and Folding Mechanisms,” Bioessays 16:199-206 (1994); and Barton, E. S., et al., “Utilization of Sialic Acid as a Coreceptor Enhances Reovirus Attachment by Multistep Adhesion Strengthening,” J. Biol. Chem. 276:2200-2211 (2000), each of which is incorporated by reference in its entirety. VTC-MS1 cell binding can be measured by FACS analysis. Washed HeLa or L-cells (3×10⁴ cells) can be incubated with or without 10-50 μg pσ1 fusion protein or pσ1 (without fused antigenic polypeptide(s)) for 30 minutes on ice. Following a wash, rabbit polyclonal anti-pσ1 or commercially prepared polyclonal Ab (against the antigen polypeptide(s) or isotype control Ab can be incubated for 30 minutes on ice. Following wash, FITC-labeled goat-anti-rabbit IgG (Jackson ImmunoResearch Laboratories) can be incubated for 30 minutes on ice. Following wash, cells can be read on flow cytometry to measure extent of bound pσ1 fusion protein. VTC-MS1 binds HeLa and L-cells with similar affinity as MOG-pσ1 and pσ1 proteins, suggesting that the head and tail/shaft domains of pσ1 in VTC-MS1 are functional (FIG. 4).

As an alternative strategy, a tripartite cloning strategy can be applied to pPICZ (Invitrogen®) expression vector for use in the yeast Pichia pastoris. In our experience, having a choice between E. coli and P. pastoris significantly increases the chance of successful expressing greater than 10 mg of the recombinant protein.

Expected results: It is anticipated that the above strategy can produce over 10 mg of purified fusion protein, such as illustrated in Table 1, with >98% purity with binding activity to L and HELA cells comparable to pσ1.

### Example 4

Introduction: This section describes an exemplary approach for determining the optimal oral dose of a fusion protein produced as described in Example 3. This is described in the context of fusion proteins that incorporate gluten polypeptides with the pσ1 targeting polypeptide, although it will be understood that the protocol can be readily modified to address dose optimization of any fusion protein described herein.

Experimental Design: Determination of the optimal oral dose of a pσ1 fusion protein comprising the gluten polypeptides is described. The study can also include control animals that are dosed with the individual fusion components, MS polypeptides and pσ1, to demonstrate that such proteins do not generate efficacy at the highest dose of VTC-MS1 fusion protein used.

Determination of the optimal oral dose of the VTC-MS1 fusion protein: To establish experimental autoimmune encephalomyelitis EAE, mice will be challenged s.c. in the flank with 150 μg of MOG₃₅₋₅₅ peptide in CFA (Sigma-Aldrich) containing 4 mg/ml Mycobacterium tuberculosis (Difco Laboratories, Detroit, Mich., USA) on day 0 (see Correale J, Farez M, Gilmore W. Vaccines for multiple sclerosis: progress to date. CNS Drugs. 2008; 22(3):175-198; Gold R, Linington C, Lassmann H. Understanding pathogenesis and therapy of multiple sclerosis via animal models: 70 years of merits and culprits in experimental autoimmune encephalomyelitis research. Brain. 2006; 129 (Pt 8):1953-1971.; Kuchroo V K, Anderson A C, Waldner H, Munder M, Bettelli E, Nicholson L B. T cell response in experimental autoimmune encephalomyelitis (EAE): role of self and cross-reactive antigens in shaping, tuning, and regulating the autopathogenic T cell repertoire. Annu Rev Immunol. 2002; 20:101-123; Bettelli E, Carrier Y, Gao W, Korn T, Strom T B, Oukka M, Weiner H L, Kuchroo V K. Reciprocal developmental pathways for the generation of pathogenic effector TH17 and regulatory T cells. Nature. 2006; 441(7090):235-238.) On days 0 and 2, mice will receive a 200 ng i.p. dose of Bordetella pertussis toxin (List Biological Laboratories; Campbell, Calif.). Mice will then be monitored and scored daily for disease progression following the standard clinical scale. Groups of 10 mice will be treated orally with PBS, increasing doses of VTC-MS1 at 5, 25, 50, 100 μg, or recombinant MOG (rMOG, as control) per mouse 10 days prior to challenge in the EAE model. We will evaluate the Clinical Score for all mice over a 30 days period based on the following score criteria (0—No clinical symptoms; 1—Limp tail; 2—Hind Limb Weakness; 3—Hind Limb Paresis; 4—Quadriplegia; 5—death).

Number of FoxP3+ T cells: The number of CD4+CD25+FoxP3+, and CD4+CD25+Foxp3− T cells present in the spleen, HNLN, MLNs, and PPs can be evaluated using flow cytometric analysis. CD4+ T cells from spleen, HNLN, MLNs, and PPs can purified by use of an automated magnetic activated cell sorter (AutoMACS) system (Miltenyi Biotec, Auburn, Calif.). The purified CD4+ T cell fraction can then be suspended in complete RPMI 1640 (4×10⁶ cells/ml) and incubated with labeled monoclonal antibodies to CD25 and FoxP3. The cells numbers can then be calculated from the flow cytometric analysis to determine the number of Foxp3+ cells.

T cell Responses: CD4+ T cells from spleen, MLNs, and PPs can purified by use of an automated magnetic activated cell sorter (AutoMACS) system (Miltenyi Biotec, Auburn, Calif.). The purified CD4+ T cell fraction can then be suspended in complete RPMI 1640 (4×106 cells/ml) and cultured with or without one mg/ml of the auto-antigenic polypeptides (for example MOG₃₅₋₅₅) in the presence of T cell-depleted, irradiated (3000 rad) splenic antigen-presenting cells (APCs) taken from non-immunized mice for five days. To assess polypeptide-specific T cell proliferative responses, an aliquot of 0.5 μCi of tritiated [3H]-TdR (Amersham Biosciences, Arlington Heights, Ill.) can be added during the final 18 hr of incubation, and the amount of [3H]-TdR incorporation can be determined by scintillation counting. The supernatants of identically treated T cell cultures not incubated with [3H]-TdR can then subjected to a cytokine-specific ELISA as described below.

T cell Cytokine Production: The cytokines and levels produced by CD4+ T cells restimulated in vitro with the MS autoantigens (for example MOG₃₅₋₅₅) can be measured by an ELISA assay. The ELISA for measuring IFN-γ, IL-2, IL-4, IL-5, IL-6, IL-10, IL-17, IL-28, and TGF-β1 can be purchased from various vendors. The levels of Ag-specific cytokine production can be calculated by subtracting the results of control cultures (e.g., without Ag stimulation) from those of Ag-stimulated cultures.

Expected Outcome: It is expected that the optimal effective dose of VTC-MS1 fusion protein will be 25 or 50 μg. It is also anticipated that efficacy will be specific to the fusion protein VTC-MS1, and neither rMOG nor pσ1 alone will have any effect on disease or CD4+ profiles, even at the highest dose of 100 μg. We also expect there to be an increased number of CD4+FoxP3+ T cells in the spleen, CNS, and HNLN in animals without symptoms of EAE, compared to control animals with EAE. We expect tolerance to the MS autoantigens will be observed through anti-inflammatory cytokine production, which includes IL-4, IL-10 and IL-28.

Alternative Strategies: If needed, the sensitization protocol described above can be adjusted by varying the number, size, and composition of the antigen dose, as well as the amount of M. tuberculosis adjuvant. In addition, it is also possible that one administration of VTC-MS1 is not sufficient to induce statistically significant tolerance at any doses tested. If this is the case, we will either increase the dose or number of VTC-MS1 treatments at the planned doses to determine the optimal prophylactic dose.

While the preferred embodiments of the compositions and methods for tolerization have been illustrated and described, it will be appreciated that various changes can be made therein without departing from the spirit and scope of the invention.

The embodiments of the invention in which an exclusive property or privilege is claimed are defined as follows:

1. An isolated fusion protein comprising a reovirus-derived targeting polypeptide, at least one polypeptide, and at least one cleavable linker.

2. The isolated fusion protein of claim 1, wherein the reovirus-derived targeting polypeptide comprises the protein sigma polypeptide (pσ1), or functional portions or derivatives thereof.

3. The isolated fusion protein of claim 1, wherein the functional portions of the pσ1 include the head domain, trimerization domain, sialic acid binding domain, and/or the shaft domain of the pσ1 protein, or any derivative thereof.

4. The isolated fusion protein of claim 1, wherein the at least one polypeptide comprises at least one epitope from a food allergen, an environmental allergen, an auto-antigen, and/or a biological therapeutic, and/or at least one epitope derived therefrom.

5. The isolated fusion protein of claim 4, wherein the food allergen is from a ground nut, tree nut, milk, gluten, egg, fish, shellfish, and the like.

6. The isolated fusion protein of claim 5, wherein the food allergen is from a peanut and the allergen polypeptide is Arah2, Arah6, Arah1, Arah3, Arah4, Arah5, Arah7, Arah8, Arah9, Arah10, Arah11, Arah12, or is derived therefrom.

7. The isolated fusion protein of claim 5, wherein the food allergen is from gluten and the allergen polypeptide is a prolamin (such as a α-gliadin, β-gliadin, γ-gliadin, ω-gliadin, hordein, secalin, zein, kafirin, avenin), glutenin, or is derived therefrom.

8. The isolated fusion protein of claim 5, wherein the food allergen is from milk and the allergen polypeptide is alpha S1-casein, alpha S2-casein, b-lactoglobulin, b-casein, k-casein, or is derived therefrom.

9. The isolated fusion protein of claim 5, wherein the food allergen is from egg and the allergen polypeptide is ovomucoid, ovotransferrin, lysozyme, livetin, apovitillin, phosvitin, or is derived therefrom.

10. The isolated fusion protein of claim 5, wherein the food allergen is from fish and the allergen polypeptide is Che ag, Lop pi, Gelatin/Ore a, Parvalbumin/Seb m, Ore a1, Seb m1, Sar sa1.0101, Albumin/Onc ma, glyceraldehyde-3-phosphate dehydrogenase, or is derived therefrom.

11. The isolated fusion protein of claim 4, wherein the environmental allergen is from an animal or insect, such as dust mite, bee, wasp, cat, dog, and the like, or plant, such as ragweed, grass, tree, and the like.

12. The isolated fusion protein of claim 11, wherein the environmental allergen is from dust mite and the allergen polypeptide is Derp1 through Derp23, Derf1 through Derf33, Eurm1, 2, 3, 4, or 14, Derm1, or is derived therefrom.

13. The isolated fusion protein of claim 11, wherein the environmental allergen is from cat and the allergen polypeptide is a secretoglobin such as Fel d 1, a lipocalin such as Feld4, an albumin such as Feld2, a cystatin such as Feld3, IgA such as Feld5w, or is derived therefrom.

14. The isolated fusion protein of claim 11, wherein the environmental allergen is from ragweed and the allergen polypeptide is Amba1 through Amba11, Ambp5, Ambt5, or is derived therefrom.

15. The isolated fusion protein of claim 11, wherein the environmental allergen is from tree, such as birch, alder, and ash, and the allergen polypeptide is Betv1, Betv2, Betv3, Betv4, Betv6, Betv7, Alng1, Alng4, Frae1, or is derived therefrom.

16. The isolated fusion protein of claim 4, wherein the autoantigen is transglutaminase, myelin-associated glycoprotein (MAG), CNS-specific myelin oligodendrocyte glycoprotein (MOG), myelin basic protein (MBP), proteolipid protein (PLP), Zinc transporter-8 (ZnT8), Glutamic decarboxylase 65 (GAD65), Glutamic decarboxylase 67 (GAD67), Preproinsulin, proinsulin, insulin, Tyrosine phosphatase like autoantigen, insulinoma antigen-2 (IA-2; ICA512, PTPRN), IA-2b (Phogrin, PTPRN2), Islet cell antigen-69 (ICA69), Chromogranin A, Islet amyloid polypeptide (ppIAPP), Heat shock protein 60 (hsp60), or is derived therefrom.

17. The isolated fusion protein of claim 4, wherein the at least one polypeptide is derived from a protein therapeutic, such as an antibody CDR or erythropoietin.

18. The isolated fusion protein of claim 1, wherein the at least one polypeptide comprises an MHC Class I epitope and/or an MHC Class II epitope.

19. The isolated fusion protein of claim 1, wherein the targeting polypeptide is separated from the at least one polypeptide by a linker.

20. The isolated fusion protein of claim 19, wherein the linker is a cleavable linker.

21. The isolated fusion protein of claim 20, wherein the cleavable linker is susceptible to cleavage by a protease present within a target cell.

22. The isolated fusion protein of claim 1, wherein the fusion protein comprises at least two polypeptides.

23. The isolated fusion protein of claim 22, wherein at least two of the polypeptides are separated by a cleavable linker.

24. The isolated fusion protein of claim 23, wherein the cleavable linker is susceptible to cleavage by a protease present within a target cell.

25. The isolated fusion protein of claim 21 or claim 24, wherein the protease is cathepsin B and/or cathepsin S.

26. The isolated fusion protein of claim 25, wherein the cleavable linker comprises an amino acid sequence GAGGVG (SEQ ID NO:1), GVGGTP (SEQ ID NO:2), or both.

27. The isolated fusion protein of claim 26, wherein the cleavable linker comprises an amino acid sequence GAGGVGGTP (SEQ ID NO:3).

28. A pharmaceutical composition comprising the isolated fusion protein of any one of claims 1-27 and a pharmaceutically acceptable carrier.

29. The pharmaceutical composition of claim 28, wherein the composition is formulated for oral or intranasal administration.

30. A nucleic acid comprising a sequence encoding the isolated fusion protein of any one of claims 1-27.

31. A vector comprising the nucleic acid of claim 30.

32. A cultured cell transfected with the vector of claim 31.

33. A method for inducing tolerance to a polypeptide, comprising administering to a subject in need thereof a pharmaceutically effective amount of the isolated fusion protein of any one of claims 1-27, wherein the isolated fusion protein comprises a polypeptide derived from the polypeptide.

34. The method of claim 33, wherein the method consists of administering a single dose of the effective amount of the isolated fusion polypeptide.

35. The method of claim 33, wherein the method comprises of administering two or more doses of the effective amount of the isolated fusion polypeptide.

36. The method of claim 33, wherein the effective amount of the isolated fusion polypeptide of claim 1, comprises less than about 100 mg, 75 mg, 50 mg, 25 mg, 20 mg, 15 mg, 10 mg, 9 mg, 8 mg, 7 mg, 6 mg, 5 mg, 4 mg, 3 mg, 2 mg, 1.5 mg, or 1 mg, of the isolated fusion polypeptide.
